(19) United States
(12) Patent Application Publication
(10) Pub. No.: US 2026/0109720 A1
(43) Pub. Date: Apr. 23, 2026

(54) Complexes and Compounds of Mercaptophenols and Curcumin for Therapeutic Applications

(71) Applicant: SUPRATIM GHOSH, KOLKATA (IN)

(72) Inventor: SUPRATIM GHOSH, KOLKATA (IN)

(73) Assignee: SUPRATIM GHOSH, KOLKATA (IN)

(21) Appl. No.: 19/428,109

(22) Filed: Dec. 20, 2025

Related U.S. Application Data
(63) Continuation-in-part of application No. PCT/IN2024/050890, filed on Jun. 22, 2024.

(30) Foreign Application Priority Data
Jun. 22, 2023 (IN) 202331041707

(51) Int. Cl. C07F 3/12; A61K 31/10; A61K 31/165; A61K 31/28; A61K 31/305; A61K 47/52; A61P 35/00; C07C 313/06; C07C 381/00; C07C 391/00; C07F 13/00

(52) U.S. Cl. CPC C07F 3/12; A61K 31/10; A61K 31/165; A61K 31/28; A61K 31/305; A61K 47/52; A61P 35/00; C07C 313/06; C07C 381/00; C07C 391/00; C07F 13/00; C07F 13/005

(57) ABSTRACT

Complexes and compounds of phenolic molecules possessing two or more ionizable groups, including curcumin, mercaptophenols, and structurally related derivatives, are disclosed. In certain embodiments, a metal or metalloid or nonmetallic atom is directly bonded to the α-carbon of curcumin or exclusively to the sulfur atom of a mercaptophenol, conferring enhanced aqueous solubility and stability under physiological and alkaline conditions. The invention includes compositions, processes for preparation, and therapeutic methods employing such complexes and compounds, which may form pharmaceutically acceptable salts suitable for biomedical applications, including parenteral or systemic administration. The complexes and compounds exhibit antiproliferative, anti-migratory, anti-invasive, and immune-stimulatory activities in cell-based and animal models and are suitable for treatment or management of conditions involving abnormal cell proliferation, pathogenic invasion, or dysregulated immune responses.

### CROSS-REFERENCE

This application is a Continuation-in-Part (CIP) Bypass under 35 U.S.C. §§ 120 and 365 (c) of International Application No. PCT/IN2024/050890, filed Jun. 22, 2024, which itself claims priority to Indian Patent Application number 202331041707, filed Jun. 22, 2023. The entire contents of the above-referenced applications are incorporated herein by reference in their entirety.

### FIELD OF THE INVENTION

The invention relates to medicinal chemistry, particularly to complexes and compounds of phenolic molecules possessing two or more ionizable groups, including curcumin, mercaptophenols, and structurally related compounds, together with their preparation and therapeutic applications.

### BACKGROUND OF THE INVENTION

Disorders characterized by abnormal cellular proliferation, dysregulated immune responses, oxidative stress, metabolic imbalance, or pathogenic invasion remain major health challenges across the globe. Despite significant advances, existing chemotherapeutics and small-molecule agents often suffer from toxicity, poor selectivity, low aqueous solubility, and reduced overall efficacy. These limitations motivate the development of new drug candidates with better therapeutic performance.

Metals and metalloids have played important roles throughout medicinal chemistry, both historically and in modern therapeutics. Clinically approved examples such as cisplatin, carboplatin, arsenic trioxide, and radioisotope-based agents demonstrate that metal-containing molecules can exert profound biological activities. However, the therapeutic use of metal-based drugs is also constrained by intrinsic toxicity, uncontrolled reactivity, and lack of selectivity. The chemical form of the metal, including its oxidation state, coordination environment, ligand sphere, and the nature of its covalent or ionic bonding, dictates both its therapeutic potential and toxicity profile. Designing metal complexes and compounds that retain beneficial activities while reducing toxicities is therefore a continuing priority in medicinal chemistry.

Phenolic molecules with conjugated, sp²-hybridized structures such as curcumin and related phenolic analogues exhibit diverse biological activities, including anti-inflammatory, antiproliferative, pro-apoptotic, and antioxidant properties. Their reactive functional groups, including consecutive keto/enol systems and ionizable hydroxyl groups, offer opportunities for chemical modification or metal coordination. Numerous curcumin-metal complexes have been described, often involving coordination through the β-diketo/dienolic moieties. These include complexes of vanadium, chromium, manganese, iron, cobalt, nickel, copper, zinc, germanium, selenium, platinum, mercury, indium, gallium, and other metals or metalloids. Despite extensive investigation, many known curcumin-metal complexes suffer from limited aqueous solubility, instability under physiological or alkaline pH for pharmacological salt formation, or require non-biocompatible solubilizers. Furthermore, many reported complexes or compounds lack desired therapeutic activity upon structural modification.

Curcumin itself is chemically unstable under alkaline pH due to rapid degradation of the β-diketone system. The Applicant is not aware of any disclosure that teaches curcumin derivative that is soluble in an aqueous environment by itself or as a sodium/potassium/calcium salt at alkaline pH while remaining stable for therapeutic applications. Although organic substituents at the α-carbon of curcumin have been previously reported, existing references reviewed by the Applicant fail to disclose any curcumin derivative in which a metal or metalloid is directly bonded to the α-carbon atom, the carbon positioned between consecutive keto/enol groups. Such direct metal-carbon bonding, absent in the art, can impart substantial stabilization to the central carbon framework, yielding improved aqueous solubility and stability at alkaline pH for therapeutic salt formation.

Mercaptophenols, including 2-, 3-, and 4-hydroxythiophenol isomers, represent another class of conjugated phenolic molecules possessing two ionizable functional groups. However, mercaptophenols are poorly soluble in aqueous environments, and their therapeutic applications have been minimally explored. Existing reports mainly describe metal complexes in which both the thiol sulfur and hydroxyl oxygen coordinate simultaneously to the same metal centre, forming bidentate chelates that often exhibit low aqueous solubility. No prior art discloses mercaptophenol complexes or derivatives featuring exclusive sulfur coordination, wherein the remaining valencies or oxidation sites of the coordinated atom are satisfied by halogens, hydroxyl, amino, thiol, acetate, carbonate, sulfate, phosphate or nitrate group/s.

Furthermore, no known reference to the Applicant describe dimeric, or polymeric mercaptophenol derivatives in which two or more mercaptophenol molecules possessing conjugated sp²-hybridized systems are linked through disulfide, amino, or oxy linkers enhancing their aqueous solubility and making them suitable for biomedical applications. The absence of disclosures of sulfur-only coordinated complexes, or aqueous-soluble derivatives of mercaptophenol, represents a significant gap in existing chemical and biomedical knowledge.

Despite recognition of these opportunities, prior disclosures reviewed by the Applicant do not describe metal or metalloid complexes in which the metal is directly bonded to the α-carbon of curcumin, neither mercaptophenol complexes exhibiting exclusive sulfur-only coordination, nor polymeric mercaptophenol derivatives suitable for biomedical use. Likewise, prior art does not report complexes or derivatives demonstrating simultaneous aqueous solubility, alkaline stability, salt-forming capability, and therapeutic efficacy across multiple cell types.

Accordingly, there remains a significant need for structurally defined, aqueous-soluble, chemically stable, and therapeutically efficacious complexes or compounds of curcumin, mercaptophenols, and related conjugated phenolic systems. The development of such complexes and compounds may overcome limitations of existing phenolic derivatives, expand therapeutic possibilities, and provide new pharmacological modalities targeting diseases associated with oxidative stress, uncontrolled cell proliferation, invasion, and immune dysregulation.

### Reservation

The foregoing description is provided to place aspects of the invention in context and is not an admission that any statement or any cited or uncited reference constitutes prior art under applicable law.

### SUMMARY OF THE INVENTION

The present invention provides complexes and compounds of phenolic molecules possessing two or more ionizable groups, including curcumin, mercaptophenols, and structurally related derivatives. Such complexes and compounds exhibit increased aqueous solubility, improved stability suitable for pharmacological preparation, and enhanced therapeutic efficacy, facilitating their use in biomedical and pharmaceutical applications.

In one aspect, the invention provides curcumin-metal compounds in which a metal or metalloid atom is directly bonded to the α-carbon of curcumin, the carbon positioned between two consecutive keto and/or enol groups. This direct metal-carbon bond stabilizes the curcumin framework against nucleophilic and electrophilic degradation at alkaline pH in aqueous media and permits the formation of sodium, potassium, calcium, or other pharmaceutically acceptable salts. These compounds exist in stabilized β-dienolic/β-diketo forms under physiological or alkaline conditions and are suitable for parenteral or systemic administration, delivering improved therapeutic efficacy.

In another aspect, the invention provides mercaptophenol complexes and derivatives in which a metal or metalloid or nonmetal atom is bonded exclusively to the thiol sulfur of a mercaptophenol molecule, without concurrent coordination to the hydroxyl oxygen of the same molecule. The remaining valencies or oxidation sites of the bonded atom may be satisfied by halogens, hydroxyl-, amino-, thiol-, carbonate-, acetate-, sulfate-, nitrate- and phosphate-groups, enabling the formation of monomeric, dimeric, or polymeric species. These complexes or derivatives exhibit significant aqueous solubility, stability, and therapeutic activity across multiple cell types.

The invention further provides methods for preparing the disclosed complexes and compounds. Curcumin compounds may be formed by reacting curcumin or related phenolic molecules with metal salts under alkaline or amphipathic conditions to generate intermediates that undergo metal-carbon bond formation. Mercaptophenol complexes and derivatives may be synthesized by generating reactive thiolate intermediates followed by reaction with metal/metalloid salts or nonmetallic chemical groups; alternatively, by allowing suitable metal/metalloid salts or nonmetallic chemical groups to dissociate and react with the thiol group to form mercaptophenol complexes and derivatives.

The disclosed complexes or compounds exhibit favorable pharmacological profiles, including enhanced aqueous solubility, stability, and preferential cytotoxicity toward malignant cells with minimal toxicity toward healthy cells. Curcumin-metal compounds demonstrate antiproliferative, antimigratory, and pro-apoptotic activity in leukemia, breast cancer, and other cell lines, while mercaptophenol complexes and derivatives show activity in leukemia and epithelial cancer models with limited effects on non-malignant cells.

The invention further provides pharmaceutical compositions, formulations, dosage forms, and methods of treatment, including administration of the complexes or compounds to subjects in need thereof for the treatment or management of cancer, infectious diseases, inflammatory disorders, or other conditions associated with uncontrolled cell proliferation, invasion, and immune dysregulation. Overall, the invention provides structurally defined curcumin compounds featuring direct α-carbon-metal bonding and mercaptophenol complexes and derivatives featuring exclusive sulfur coordination with therapeutic applicability, representing a class of pharmaceutical candidates with significant potential for drug development.

### BRIEF DESCRIPTION OF DRAWINGS

FIG. 1 illustrates the chemical structure of a class of organometallic compounds of curcumin, showing a metal or metalloid atom is directly bonded to the α-carbon atom between two ionizable keto/enol groups, wherein the other coordination sites of the metal or metalloid atom is satisfied by chemical groups/linkers as represented by Formula I.

FIG. 2 illustrates the chemical structure of a class of complexes and derivatives of mercaptophenol in which a metal or metalloid or non-metallic atom is exclusively bonded to the sulfur atom of the thiol group without concurrent bonding to the hydroxyl oxygen, wherein the other coordination sites of the bonded atom is satisfied by chemical groups/linkers, as represented by Formula II.

FIG. 3 illustrates the chemical structure of a curcumin-mercury compound, prepared according to the bonding strategy of Formula I.

FIG. 4 illustrates the UV-visible spectrum of the curcumin-mercury compound in 10 mM NaOH, showing characteristic absorption peaks.

FIG. 5 illustrates the fluorescence emission spectrum of the curcumin-mercury compound in ethanol containing 10 mM NaOH.

FIG. 6 illustrates the ESI-MS spectrum of the curcumin-mercury compound.

FIG. 7 illustrates the ¹H NMR spectrum of the curcumin-mercury compound recorded in d₆-DMSO.

FIG. 8 illustrates the ¹³C NMR spectrum of the curcumin-mercury compound recorded in d₆-DMSO.

FIG. 9 illustrates a time-dependent ¹H NMR stability study of the curcumin-mercury compound at 0, 6, and 12 hours under alkaline aqueous medium.

FIG. 10 illustrates a time-dependent UV-visible stability study of the curcumin-mercury compound at 0, 6, and 12 hours under alkaline aqueous medium.

FIG. 11 illustrates an XPS survey spectrum of the curcumin-mercury compound.

FIG. 12 illustrates XPS spectra for C1s, O1s, and Hg 4f regions of the curcumin-mercury compound.

FIG. 13 illustrates a cell-viability assay of the curcumin-mercury compound on MOLT-4, HL-60, and human PBMC cells.

FIG. 14 illustrates a scratch-migration assay of the curcumin-mercury compound on MDA-MB-231 cells.

FIG. 15 illustrates an apoptosis assay of the curcumin-mercury compound on HL-60 cells.

FIG. 16 illustrates a cell-cycle assay of the curcumin-mercury compound on MOLT-4 cells.

FIG. 17 illustrates an erythrocyte hemolysis assay of the curcumin-mercury compound using healthy human blood.

FIG. 18 illustrates representative flow-cytometry data showing expression of immunogenic markers following treatment with the curcumin-mercury compound in lymphoblasts from an ALL patient.

FIG. 19 illustrates total white blood cell counts, red blood cell counts, and differential white blood cell counts in control and compound-treated animal groups via tail-vein injection.

FIG. 20 illustrates the chemical structure of a curcumin-manganese compound prepared according to Formula I.

FIG. 21 illustrates the UV-visible spectrum of the curcumin-manganese compound.

FIG. 22 illustrates the fluorescence emission spectrum of the curcumin-manganese compound in water and ethanol.

FIG. 23 illustrates the FTIR spectrum of the curcumin-manganese compound.

FIG. 24 illustrates a cell-viability assay of the curcumin-manganese compound on MDA-MB-231 cells.

FIG. 25 illustrates a scratch-migration assay of the curcumin-manganese compound on MDA-MB-231 cells.

FIG. 26 illustrates the chemical structure of 2-mercaptophenol-mercury complex prepared according to Formula II.

FIG. 27 illustrates the UV-visible spectrum of the 2-mercaptophenol-mercury complex in 10 mM NaOH.

FIG. 28 illustrates a time-dependent UV-visible stability study of the 2-mercaptophenol-mercury complex in alkaline conditions.

FIG. 29 illustrates the ESI-MS spectrum of the 2-mercaptophenol-mercury complex.

FIG. 30 illustrates an XPS survey spectrum of the 2-mercaptophenol-mercury complex.

FIG. 31 illustrates the XPS spectrum of the sulfur region for the 2-mercaptophenol-mercury complex.

FIG. 32 illustrates the XPS spectrum of the mercury region for the 2-mercaptophenol-mercury complex.

FIG. 33 illustrates a cell-viability assay of the 2-mercaptophenol-mercury complex on HL-60 and HEK293 cells.

FIG. 34 illustrates a cell-viability assay of the 2-mercaptophenol-mercury complex on MCF7 and HEK293 cells.

FIG. 35 illustrates a scratch-migration assay of the 2-mercaptophenol-mercury complex on MDA-MB-231 cells.

FIG. 36 illustrates the chemical structure of a 2-mercaptophenol-thio-manganese (2MP-thio-manganese) complex, prepared according to the bonding strategy of Formula II.

FIG. 37 illustrates the UV-visible spectrum of the 2MP-thio-manganese complex in water, showing characteristic absorption peaks.

FIG. 38 illustrates a time-dependent UV-visible stability study of the 2MP-thio-manganese complex in water for 12 hours.

FIG. 39 illustrates the ¹H NMR spectrum of the 2MP-thio-manganese complex recorded in d₆-DMSO.

FIG. 40 illustrates the EPR spectrum of the 2MP-thio-manganese complex recorded in D₂O.

FIG. 41 illustrates an XPS survey spectrum of the 2MP-thio-manganese complex.

FIG. 42 illustrates comparative XPS spectrum of C 1s, O 1s, S 2p, Mn 2p of the 2-mercaptophenol (A, B, C) and 2MP-thio-manganese complex (D, E, F, G).

FIG. 43 illustrates the ESI-MS spectrum of the 2MP-thio-manganese complex.

FIG. 44 illustrates the FTIR spectrum of the 2MP-thio-manganese complex in D₂O.

FIG. 45 illustrates comparative Raman spectrum of the 2-mercaptophenol and 2MP-thio-manganese complex.

FIG. 46 illustrates a cell-viability assay of the 2MP-thio-manganese complex on MDA-MB-231 cells.

FIG. 47 illustrates an apoptosis assay of MDA-MB-468 cells with 2MP-thio-manganese complex treatment, via FACS analysis.

FIG. 48 illustrates cell cycle analysis of MDA-MB-231 and MDA-MB-468 cells with 2MP-thio-manganese complex treatment via PI staining and flow cytometry.

FIG. 49 illustrates a scratch-migration assay on MDA-MB-231 cells upon 2MP-thio-manganese complex treatment at 0, 12 and 24 hours.

FIG. 50 illustrates representative flow-cytometry data showing expression of immunogenic markers following treatment with the 2MP-thio-manganese complex in WBC population from healthy individuals.

FIG. 51 illustrates the chemical structure of a 2-mercaptophenol-selenium (2MP-selenium) complex, prepared according to the bonding strategy of Formula II.

FIG. 52 illustrates the UV-visible spectrum of the 2MP-selenium complex in water, showing characteristic absorption peaks.

FIG. 53 illustrates a time-dependent UV-visible stability study of the 2MP-selenium complex in water for 12 hours.

FIG. 54 illustrates the ¹H NMR spectrum of the 2MP-selenium complex recorded in de-DMSO.

FIG. 55 illustrates the ESI-MS spectrum of the 2MP-selenium complex.

FIG. 56 illustrates XPS spectrum of C 1s, O 1s, S 2p, Se 3d of the 2MP-selenium complex (A, B, C, D).

FIG. 57 illustrates a cell-viability assay of the 2MP-selenium complex on NCIH460 and A549 cells.

FIG. 58 illustrates mitochondrial trans-membrane potential assay of NCIH460 cells with 2MP-selenium complex treatment, via FACS analysis.

FIG. 59 illustrates mitochondrial trans-membrane potential assay of A549 cells with 2MP-selenium complex treatment, via FACS analysis.

FIG. 60 illustrates a scratch-migration assay on NCIH460 cells upon 2MP-selenium complex treatment at 0, 12 and 24 hours.

FIG. 61 illustrates the chemical structure of a 2-mercaptophenol-sulfoxide (2MP-sulfoxide) dimer, prepared according to the bonding strategy of Formula II.

FIG. 62 illustrates the UV-visible spectrum of the 2MP-sulfoxide dimer in water, showing characteristic absorption peaks.

FIG. 63 illustrates a time-dependent UV-visible stability study of the 2MP-sulfoxide dimer in water for 12 hours.

FIG. 64 illustrates the 1H NMR spectrum of the 2MP-sulfoxide dimer recorded in d₆-DMSO.

FIG. 65 illustrates the ESI-MS spectrum of the 2MP-sulfoxide dimer.

FIG. 66 illustrates XPS spectrum of C 1s, O 1s, S 2p of the 2MP-sulfoxide dimer (A, B, C).

FIG. 67 illustrates a cell-viability assay of the 2MP-sulfoxide dimer on NCIH460 and A549 cells.

FIG. 68 illustrates cell cycle analysis of A549 and NCIH460 cells with 2MP-sulfoxide dimer treatment via PI staining and flow cytometry.

FIG. 69 illustrates a scratch-migration assay on A549 cells upon 2MP-sulfoxide dimer treatment at 0, 12 and 24 hours.

FIG. 70 illustrates the chemical structure of a 2-mercaptophenol-sulfinamide (2MP-sulfinamide) dimer, prepared according to the bonding strategy of Formula II.

FIG. 71 illustrates the UV-visible spectrum of the 2MP-sulfinamide dimer in water, showing characteristic absorption peaks.

FIG. 72 illustrates a time-dependent UV-visible stability study of the 2MP-sulfinamide dimer in water for 12 hours.

FIG. 73 illustrates the ¹H NMR spectrum of the 2MP-sulfinamide dimer recorded in de-DMSO.

FIG. 74 illustrates an XPS survey spectrum of the 2MP-sulfinamide dimer.

FIG. 75 illustrates comparative XPS spectrum of C 1s, O 1s, S 2p, N 1s of the 2-mercaptophenol (A, B, C) and 2MP-sulfinamide dimer (D, E, F, G).

FIG. 76 illustrates the ESI-MS spectrum of the 2MP-sulfinamide dimer.

FIG. 77 illustrates the FTIR spectrum of the 2MP-sulfinamide dimer in D₂O.

FIG. 78 illustrates comparative Raman spectrum of the 2-mercaptophenol and 2MP-sulfinamide dimer.

FIG. 79 illustrates a cell-viability assay of the 2MP-sulfinamide dimer on MDA-MB-468 cells.

FIG. 80 illustrates an apoptosis assay of MDA-MB-231 cells with 2MP-sulfinamide dimer treatment, via FACS analysis.

FIG. 81 illustrates cell cycle analysis of MDA-MB-231 and MDA-MB-468 cells with 2MP-sulfinamide dimer treatment via PI staining and flow cytometry.

FIG. 82 illustrates a scratch-migration assay on MDA-MB-468 cells upon 2MP-sulfinamide dimer treatment at 0, 12 and 24 hours.

FIG. 83 illustrates representative flow-cytometry data showing expression of immunogenic markers following treatment with the 2MP-sulfinamide dimer in WBC population from healthy individuals.

### DETAILED DESCRIPTION OF THE INVENTION

### General Remarks

The invention relates to complexes and compounds of phenolic molecules having two or more ionizable groups (e.g., keto, enol, or thiol), including curcumin, mercaptophenols, and structurally related derivatives. These phenolic molecules possess sp²-hybridized conjugated systems and include atoms of suitable electronegativity, such as carbon (2.55) and sulfur (2.58) on the Pauling scale, capable of forming direct bonds with metals or metalloids. Mercaptophenols can also bond via disulfide or oxygen- or nitrogen-containing functional groups. Such bonding strategies enable the preparation of complexes and compounds exhibiting enhanced aqueous solubility, stability and therapeutic activity.

The following description is provided to facilitate understanding of illustrative embodiments of the invention. It refers to the accompanying drawings and uses specific terminology for clarity. However, the invention is not limited to the particular embodiments described, and modifications or variations apparent to persons of ordinary skill in the art are considered within the scope of the invention.

The terms “comprises,” “comprising,” and variants thereof denote non-exclusive inclusion. Thus, compositions or methods described herein may include additional elements or steps not explicitly recited. References to “an embodiment,” “another embodiment,” or similar terminology may refer to the same or different embodiments unless otherwise stated.

Unless indicated otherwise, technical and scientific terms have the meanings commonly used in the relevant art. The methods and examples described are illustrative and not limiting.

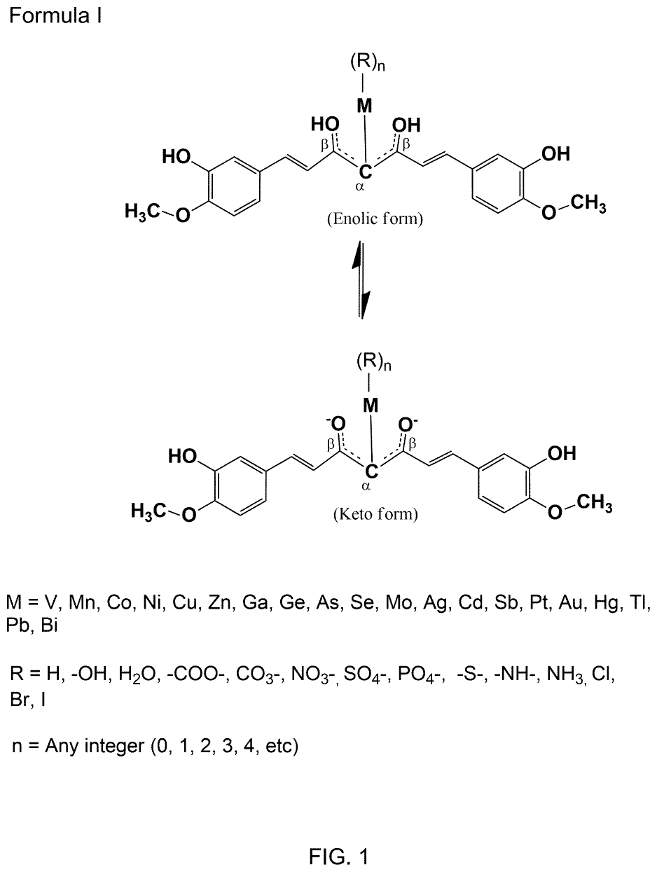

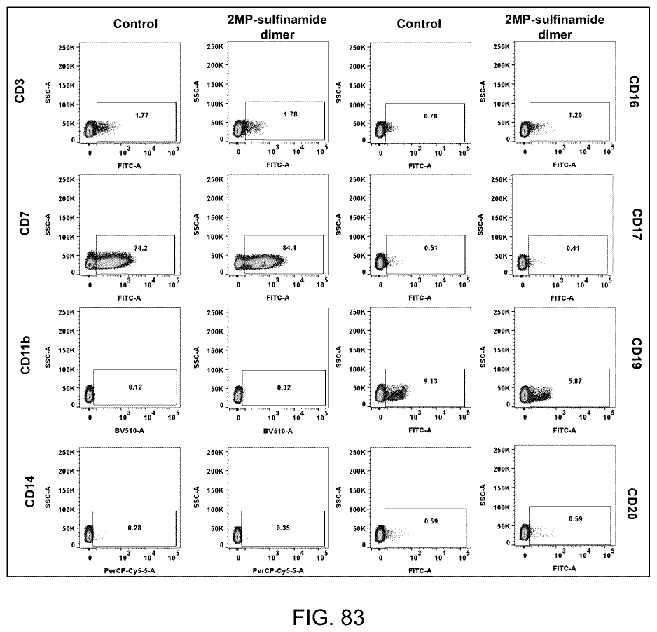

Embodiments of the invention are described with reference to the drawings, including Formula I, Formula II, and the structures and data sets presented in FIGS. 1-83.

### Measurement Conventions

### Unless Otherwise Indicated:

As used herein, solvent polarity refers to the Paul C. Sadek's The HPLC Solvent Guide.

“Aqueous solubility” for curcumin-based compounds refers to solubility of at least about 2 mM in 10 mM NaOH at 25° C., wherein the intrinsic buffering capacity of the compound adjusts the pH to about 8.2-8.5. Such stock solutions may be diluted using culture media for in vitro experimentations or used for preparation of pharmaceutical compositions for parenteral or systemic administration.

“Aqueous solubility” for mercaptophenol-containing complexes and derivatives refers to solubility in the range of about 1.5-15.0 mM in water at 25° C., with compatibility for dilution in water or physiologically relevant media.

“Kinetic stability” denotes retention of ≥95% of the principal UV-Vis absorbance (λₘₐₓ) or principal ¹H NMR resonance over approximately 12 hours at 25° C. in water, 10 mM NaOH, or biologically relevant medium.

“Antiproliferative activity” refers to a reduction in viable cell number by MTT (or equivalent) assay after ˜72 hours of treatment with the candidate complexes or compounds relative to vehicle control. Curcumin compounds typically exhibit ≥20% reduction at ˜3.2 μM; mercaptophenol complexes and derivatives exhibit ≥20% reduction in the range of 0.6-3.0 μM.

Equivalent analytical methods, measurement conditions, or solvent systems may be used.

### I. Organometallic Compounds of Curcumin (Formula I)

FIG. 1 (formula I) illustrates organometallic compounds of phenolic molecules having an sp²-hybridized or conjugated system, such as curcumin. These include curcumin, curcumin derivatives, curcumin precursors, and their metal-bound analogues. In these embodiments, a metal or metalloid atom is directly bonded to the α-carbon of curcumin and remaining valencies or oxidation sites of the metal or metalloid atom may be satisfied by halogens, oxygen-, sulfur-, carbon-, nitrogen- and phosphorus-containing functional groups or molecules. The α-carbon is positioned between two consecutive β-keto/enolic group and forms partial double bond with adjacent β-keto/enolic carbon upon direct bonding with metal or metalloid atom.

Suitable metals or metalloids (M) include, without limitation, V, Mn, Co, Ni, Cu, Zn, Ga, Ge, As, Se, Mo, Ag, Cd, Sb, Pt, Au, Hg, TI, Pb, and Bi.

Suitable valency-satisfying groups or ligands (R) include, without limitation, H, —OH, H₂O, —COO—, CO₃—, NO₃—, SO₄—, PO₄—, —SH, —NH₃, —NH₂, —NH, Cl, Br, and I.

In certain embodiments, the metal or metalloid atom is directly bonded to the α-carbon positioned between two ionizable keto/enol groups within the conjugated framework.

The α-carbon is relatively acidic and prone to nucleophilic attack following deprotonation at alkaline pH. Direct metal-carbon bonding reduces acidity at this position and stabilizes the molecule against electrophilic degradation in aqueous environments.

Compounds prepared according to Formula I predominantly exist as β-diketo species, under physiological as well as alkaline aqueous conditions and remain stable at pH 7.2-10 for at least 12 hours at room temperature (18-25° C.). Depending upon the oxidation state of the bonded metal atom, compounds prepared according to Formula I may also exist in β-dienolic form around physiological pH and remain stable for at least 12 hours at room temperature.

The compounds exhibit simultaneous aqueous solubility and stability at pH 7.2-10. They readily form pharmaceutically suitable sodium, potassium, or calcium salts and may engage in non-covalent interactions with amphipathic molecules such as nucleotides, nucleosides, oligonucleotides, and polyethylene glycol (PEG).

Stability under alkaline conditions enhances solubility and facilitates preparation of stable injectable salt forms. Partial double-bond character between the α-carbon and adjacent β-carbons contributes to salt stability at physiological temperature (37° C.) for extended periods of time.

These stabilized compounds may associate non-covalently with chemotherapeutic nucleosides and nucleotides, including 5-fluorouracil, gemcitabine, cytarabine, and derivatives, supporting combination therapy. The disclosed compounds represent the structurally defined curcumin derivatives featuring direct α-carbon-metal bonding suitable for biomedical use.

Preliminary biological studies demonstrate preferential cytotoxicity toward malignant cells, including HL-60 and MOLT-4 acute leukemia cells, at 0.5-25 UM, with minimal toxicity toward healthy PBMCs and RBCs. These properties support potential utility in cancer and other diseases involving uncontrolled proliferation and invasion of disease-causing cells and/or immune dysregulation.

### II. Complexes and Derivatives of Mercaptophenols (Formula II)

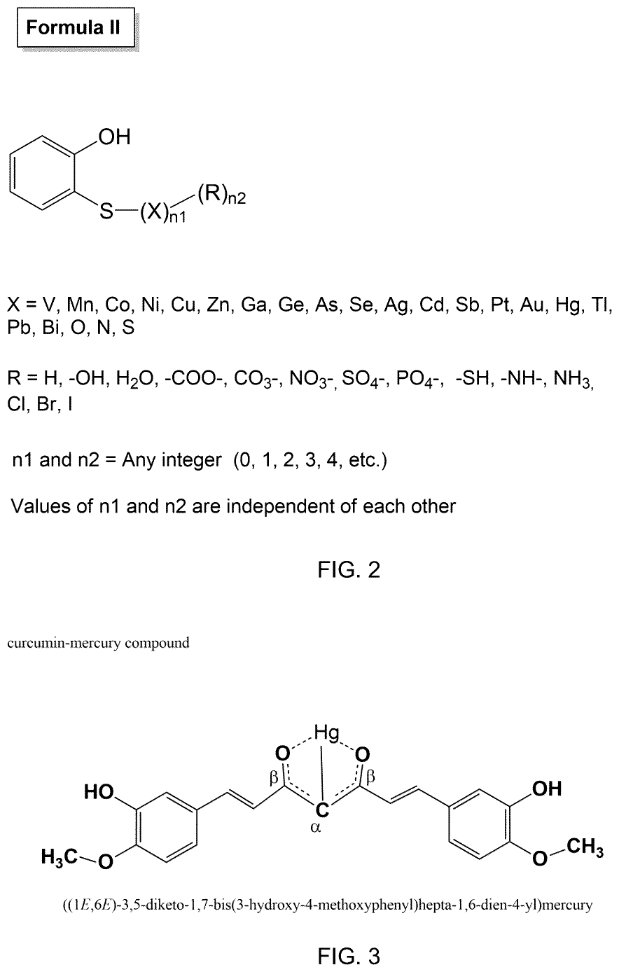

FIG. 2 (formula II) illustrates complexes and derivatives of mercaptophenols, including 2-, 3-, and 4-mercaptophenol, isomers, formed with metal or metalloid or nonmetal atoms according to Formula II.

In certain embodiments, the metal or metalloid atom or nonmetallic chemical group/linker is bonded exclusively to the sulfur atom of the mercaptophenol thiol group without concurrent bonding to the hydroxyl oxygen of the same mercaptophenol molecule. Remaining valencies or oxidation sites of the metal or metalloid atom or chemical group may be occupied by halogens or oxygen-, sulfur-, carbon-, nitrogen- and phosphorus-containing functional groups or linkers.

Suitable metals or metalloids or nonmetal atom (X) include, without limitation, V, Mn, Co, Ni, Cu, Zn, Ga, Ge, As, Se, Mo, Ag, Cd, Sb, Pt, Au, Hg, TI, Pb, Bi, O, N and S.

Suitable valency or oxidation site satisfying groups or linkers (R) include, without limitation, H, —OH, H₂O, —COO—, CO₃—, NO₃—, SO₄—, PO₄—, —SH, —NH₃, —NH₂, —NH, Cl, Br, and I.

Exclusive sulfur bonding promotes aqueous solubility and enables formation of linear derivatives, monomers, dimers, or polymers having therapeutic potential.

Two mercaptophenol molecules can also bond with each other via disulfide or sulfinamide or sulfoxide linkage to form mercaptophenol-polymer.

The complexes or derivatives exhibit aqueous solubility and stability at physiological pH. They can form chloride, bromide, iodide, sodium, potassium or calcium salts and may also conjugate or complex with amphipathic chemotherapeutic molecules. They exhibit anti-proliferative, anti-migratory, anti-invasive and immune-modulatory activities at 0.3-30 μM.

Preliminary biological evaluations demonstrate preferential activity against HL-60 leukemia cells, MCF7, MDA-MB-231, MDA-MB-468 breast cancer cells and NCIH460, A549 non-small cell lung cancer cells with minimal toxicity toward non-cancerous HEK-293 cells.

### Additional Embodiments

In additional embodiments, mercaptophenol-based complexes or derivatives may be formed in which both the sulfur atom of the thiol group and the oxygen atom of the phenolic hydroxyl group participate in coordination with a metal or metalloid center. In such embodiments, the metal or metalloid atom may further coordinate one or more additional oxygen-containing ligands, including hydroxyl, oxide, or solvent-derived ligands, thereby enhancing aqueous solubility and stability of the resulting complex or derivative.

These embodiments may give rise to monomeric, dimeric, oligomeric, or polymeric structures, including oxygen-bridged or hydroxyl-bridged assemblies, while retaining antiproliferative, anti-migratory, anti-invasive, or immune-modulatory activity. Such embodiments are considered alternative coordination modes distinct from the exclusively sulfur-bonded complexes described elsewhere herein.

These additional embodiments are disclosed for completeness of description and do not limit the scope of the claimed exclusively sulfur-bonded mercaptophenol complexes.

### III. Methods of Preparation

### A. Curcumin Organometallic Compounds

Organometallic curcumin compounds may be synthesized by reacting curcumin or related phenolic molecules with metal salts (chloride, bromide, iodide, acetate, sulfate, nitrate, phosphate) in solvents of polarity 5.0-6.0 (methanol, ethanol, propanol, acetonitrile, acetone). Curcumin may be dissolved at a concentration of 0.1-10 mM, followed by addition of water and aqueous ammonium hydroxide solution at 4-50° C., until pH reaches to 7.5-10.0 to generate a reactive intermediate. Then a metal salt may be added directly or reacted with isolated intermediates at molar ratios of about 1:0.5 to 1:50 (phenolic molecule:metal salt=1:0.5-1:50). Reaction mixtures may be incubated until color change and/or turbidity appears. Alternatively, depending upon the reactivity and solubility of the metal, metal salt may be directly added to the curcumin solution, followed by addition of aqueous ammonium hydroxide at 4-50° C., until color change and/or turbidity is observed. Reaction mixtures may then be processed by cooling, and centrifugation (e.g., 4,000-10,000 g for 10-15 minutes), washing, and drying at ≤40° C.

### B. Mercaptophenol Complexes and Derivatives

Mercaptophenol complexes or derivatives may be synthesized by dissolving mercaptophenol in a solvent of polarity 5.0-6.0 (methanol, ethanol, propanol, acetonitrile, acetone) at a concentration of 0.1-10 mM, followed by addition of water to yield a final water content of ˜1-25% (v/v). Metal or metalloid salt may be added directly to react with the mercaptophenol solution at 4-50° C., followed by incubation until color change and/or turbidity is observed. Alternatively, reaction could be performed by addition of metal or metalloid salt to the mercaptophenol solution as described previously, followed by addition of an alkaline solution (NaOH, KOH, Ca(OH)₂, or NH₄OH) dropwise, at 4-50° C., at a molar ratio of mercaptophenol:alkali=1:0.1-1:10 and incubated until color change or turbidity or both observed. Yet, in another method, synthesis may be performed by adding an alkaline solution (NaOH, KOH, Ca(OH)₂ or NH₄OH) to a mercaptophenol solution as mentioned above at 4-50° C., at a molar ratio of mercaptophenol:alkali=1:0.1-1:10 to form a thiolate intermediate; this intermediate may be reacted with a metal or metalloid salt at a molar ratio of about 1:0.5 to 1:50. After incubation for 5-60 minutes (optionally longer depending on metal and pH) until color change or turbidity or both is observed, the reaction mixture may be dried or centrifuged, and supernatant as well as pellet portions were separated and dried, followed by washing with solvents of increasing polarity, such as, n-hexane, toluene, carbon tetrachloride, dichloromethane, chloroform, methanol, ethanol and water. Washed fractions of each solvent may then be dried and desired mercaptophenol complexes or derivatives may be isolated by dissolving in aqueous solutions of suitable pH. Linear derivatives, monomer, dimer or polymer may be synthesized; especially nonmetallic mercaptophenol derivatives should be isolated from relatively less polar fractions, such as, n-hexane, toluene or dichloromethane, while organometallic complexes of mercaptophenol may be isolated from relatively polar fractions, such as, chloroform or methanol or ethanol. In all above cases metal or metalloid salt was used in 1:0.5 to 1:50 (metcaptophenol:

$$\text{Metal/metalloid} = 1 ∶ 0.5 - 1 : 50 ) \text{molar ratio} .$$

### IV. Analytical and Biological Characterization

Analytical methods, including UV-Vis, fluorescence, FTIR, Raman, ESI-MS, NMR, and XPS, confirm structural features, solubility, and stability.

### Representative Identifiers Include:

change in UV-Vis spectral pattern, along with appearance of new absorbance maxima,
disappearance of the curcumin α-H signal (˜6.0-6.2 ppm) in ¹H NMR,
shift of α-C signal (˜101 ppm to ˜96.4 ppm) in ¹³C NMR,
sulfur-binding shifts for mercaptophenol complexes and derivatives,
XPS signatures for corresponding elements,
change in XPS pattern and appearance of new bands for C_{α}-M, S-M, S—O, S—N, S—S bond formation,
ESI-MS data consistent with expected stoichiometry.

Cell-based assays, including MTT, Annexin-V/PI flow cytometry, cell-cycle analysis, and migration/invasion assays, and expression pattern of immune-markers (by FACS) demonstrate cytotoxicity, apoptosis induction, cell-cycle arrest, anti-migratory, and immune-modulation activity.

### Examples

### General Notes for Examples

The following examples illustrate specific embodiments of the invention and are not intended to limit its scope. Reaction parameters, including temperature, pH, solvent composition, metal-to-ligand ratios, incubation times, and purification conditions, may be varied or optimized by persons skilled in the art without departing from the general structure, processes, or therapeutic methods described in the claims. Analytical techniques and reagent sources may likewise be substituted with equivalent alternatives.

### Example 1: Curcumin-Mercury Compound

### IUPAC Name: ((1E,6E)-3,5-diketo-1,7-bis(3-hydroxy-4-methoxyphenyl) hepta-1,6-dien-4-yl) mercury

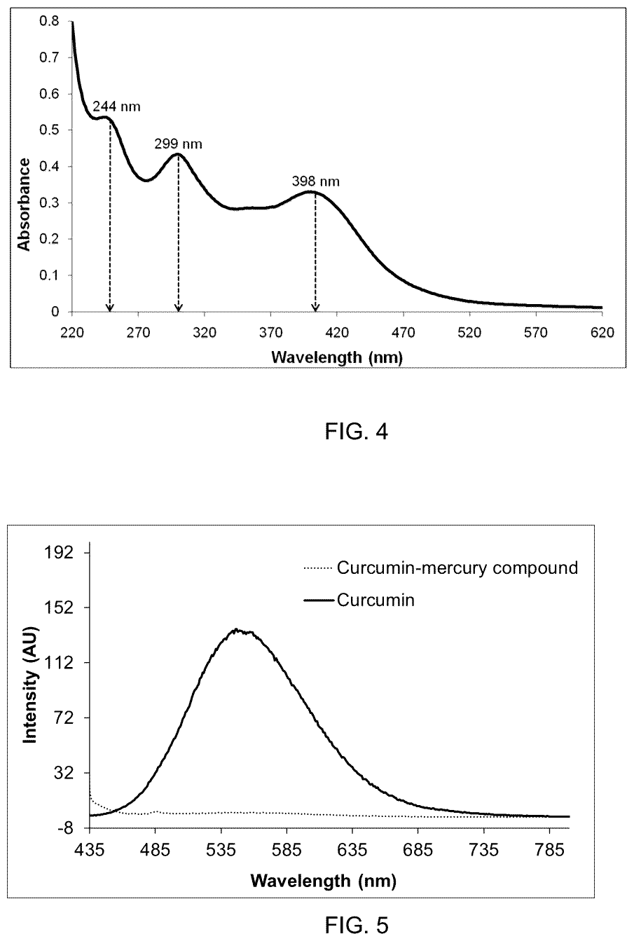

Organometallic compound of curcumin as claimed in claim 1 and represented in FIG. 1 (formula I) and FIG. 4 is synthesized as follows. Curcumin (1,7-bis(4-hydroxy-3-methoxyphenyl) hepta-1,6-diene-3,5-dione) was dissolved in anhydrous ethanol and stirred overnight. Water was added to adjust the final curcumin concentration to 1 mM and final water content ˜5% (v/v). The solution was transferred to a round-bottom flask and stirred at room temperature while ammonium hydroxide was added until pH 7.5-8.5 was reached. Mercuric chloride (HgCl₂) was then added at a curcumin: HgCl₂ molar ratio of 1:12. The reaction mixture was incubated for 4-5 hours at ˜25° C. until turbidity appeared.

The mixture was divided into centrifuge tubes and centrifuged at 10,650×g for 10 minutes at 18° C. The supernatant was discarded, and pellets were washed repeatedly with anhydrous ethanol with intermittent sonication. After additional centrifugation and washing, the pellet was resuspended in water and centrifuged again. The final pellet was dried at room temperature or lyophilized. For experiments, the dried material was dissolved in DMSO or 10 mM NaOH; if slight turbidity was present, the suspension was clarified by centrifugation and the colored supernatant was used.

### Experimental Characterization and Results

UV-Vis spectra of curcumin-mercury compound (compound dissolved in 10 mM NaOH) in FIG. 4 depicts peaks at 398, 299, and 244 nm. The characteristic curcumin peak at ˜426 nm shifted hypsochromically to 398 nm, and the n→π* transition shifted from ˜260 nm to 244 nm, consistent with metal bonding to the carbon frame.

Fluorescence spectra (ethanol containing NaOH) depicted in FIG. 5 showed quenching of curcumin fluorescence, indicating binding of mercury.

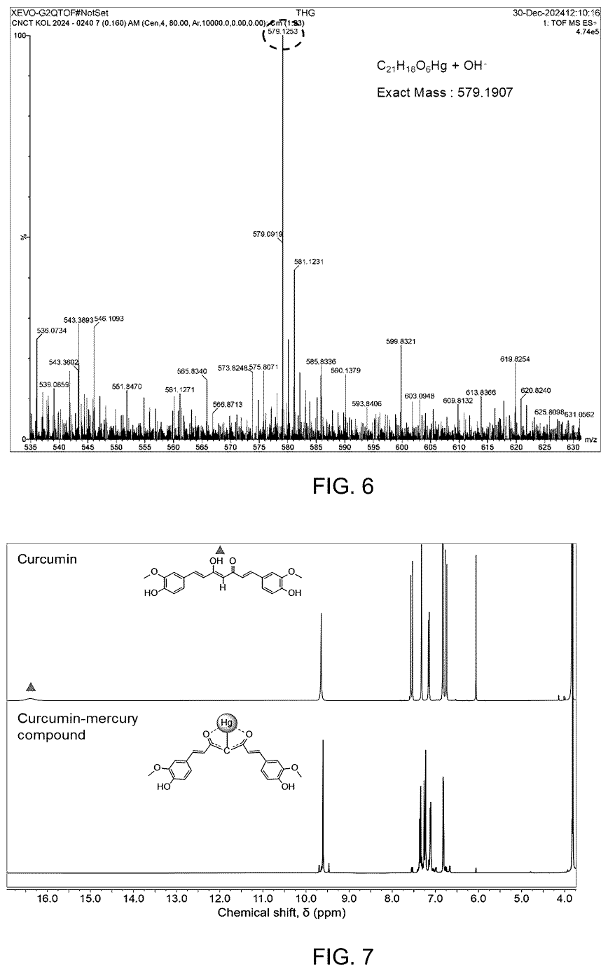

Mass spectrometry as shown in FIG. 6 demonstrated a dominant ion at m/z 579.1253, in agreement with the expected mass as hydroxyl adduct.

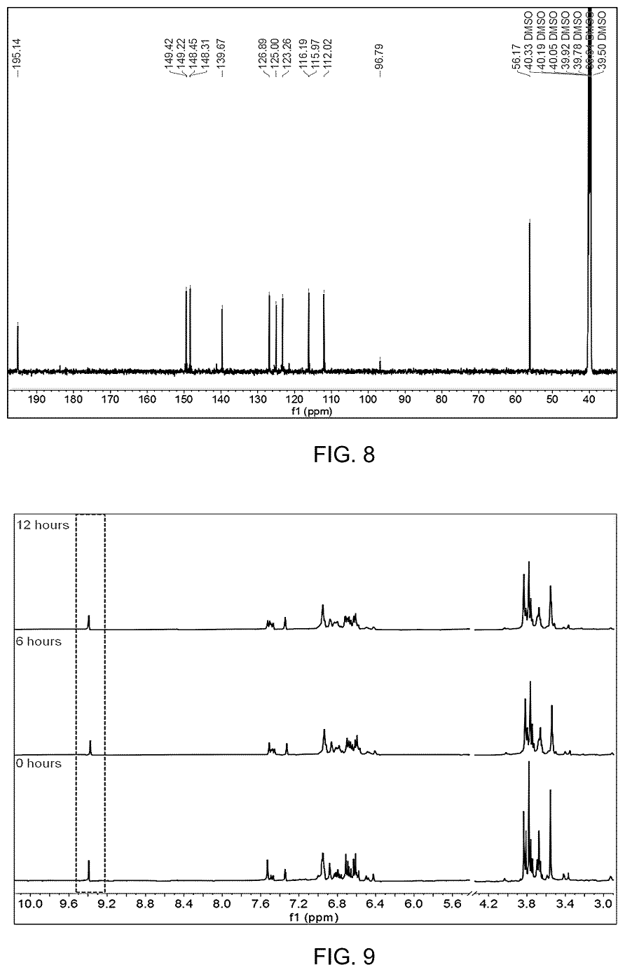

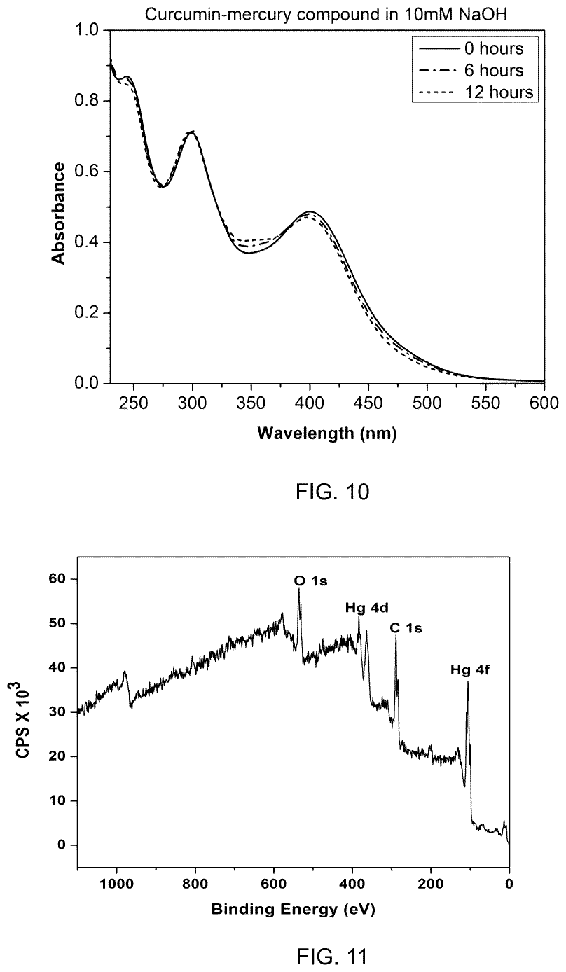

¹H and ¹³C NMR as shown in FIGS. 7 and 8, respectively, demonstrated loss of the α-proton signal at 6.06 ppm, confirming α-carbon substitution. Methoxy and phenolic OH signals appeared at 3.81 and 9.61 ppm, respectively. Carbon signals between 50-200 ppm, including those near 195 ppm, were consistent with β-diketo curcumin. The shift of C_{α} band from 101 to 96.8 ppm further confirmed the delocalization of electron cloud upon direct metal binding to the C_{α} of curcumin. The compound remained stable as sodium salt with 10 mM NaOH in water for at least 12 hours at room temperature, as shown in FIGS. 9 and 10.

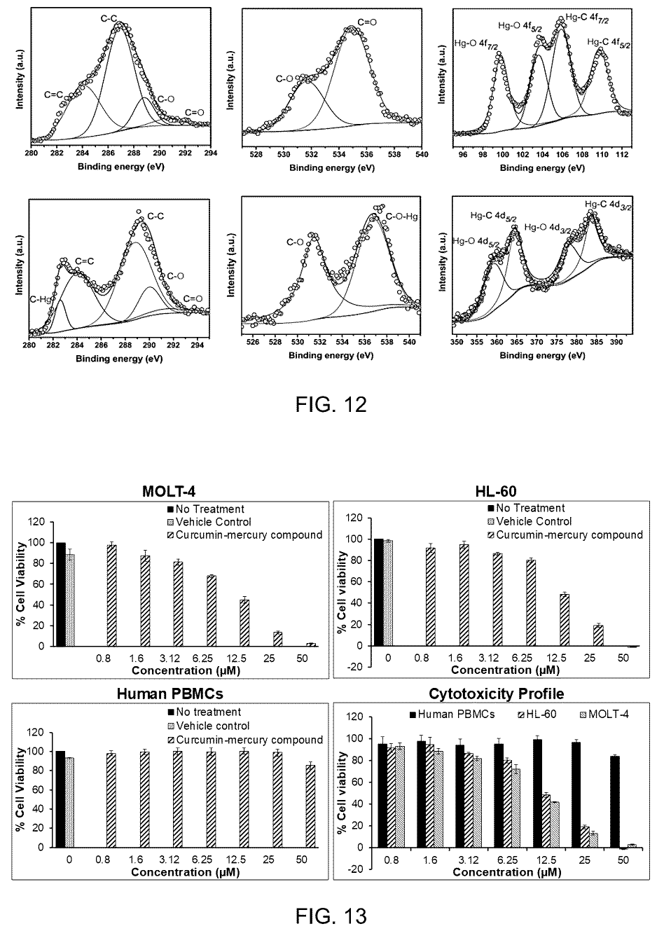

As depicted in FIG. 11, XPS spectrum demonstrated presence of all elements of the curcumin-mercury compound. FIG. 12 is showing the XPS spectrum of carbon (C 1s) of curcumin is deconvoluted to four peaks for sp2 carbon, sp3 carbon, C—O and C═O; however, deconvolution of carbon (C 1s) spectrum of curcumin-mercury compound demonstrated five components with significant change in spectral pattern. A new peak is deconvoluted at 282.59 eV that is assigned to C—Hg photoemission, mercury being the newly included and least electronegative bonding partner of carbon in the synthesized structure. Further, photoemission signals for C—C, C—O and C═O are shifted to higher binding energy (BE), from 286.96, 288.81 and 290.67 eV in curcumin to 288.91, 290.05 and 291.71 eV, respectively, in curcumin-mercury compound. These higher energy shifts are due to metalation of the α-carbon, affecting electron density on adjacent carbon atoms in the compound. The XPS spectra of Hg (Hg 4f and 4d) showed four intensified peaks for each, confirming the involvement of carbon atoms in bonding with mercury. Hg 4f spectra confirmed Hg (II) as a single oxidation state.

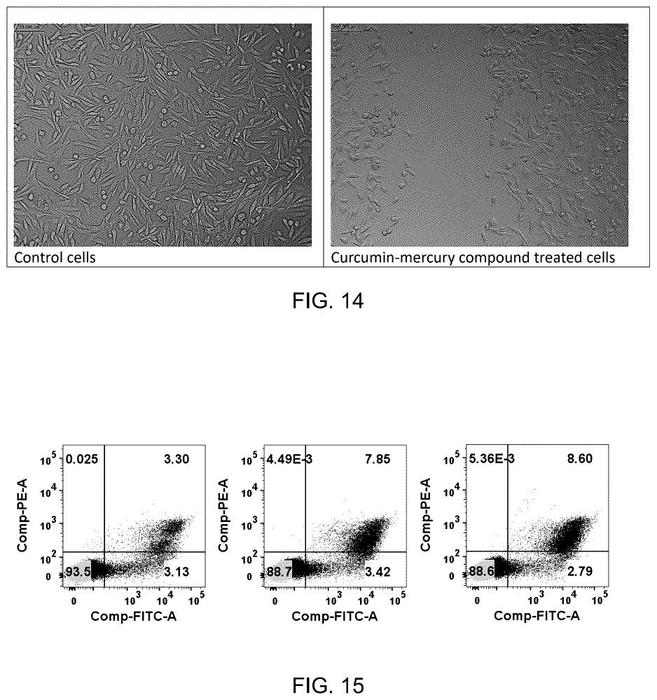

MTT assays revealed dose-dependent antiproliferative activity against MOLT-4 and HL-60 leukemia cells, with IC₅₀ values of ˜10 UM and ˜12.5 UM, respectively, as shown in FIG. 13. PBMCs showed no measurable toxicity. Scratch assays demonstrated inhibition of MDA-MB-231 breast-cancer cell migration, corresponding micrographs are shown in FIG. 14.

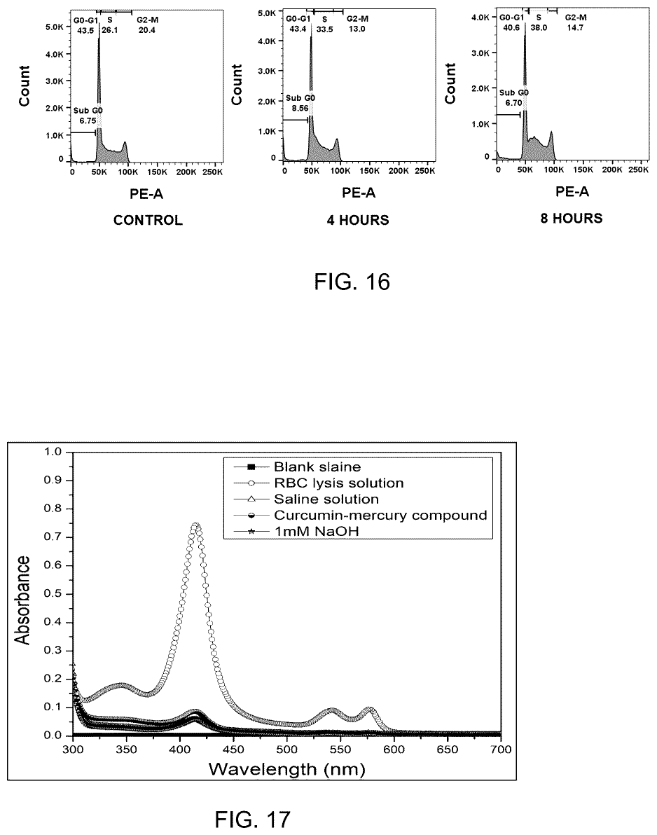

As shown in FIG. 15, Annexin-V/PI staining (HL-60 cells) showed time-dependent apoptosis without necrosis. PI cell-cycle analysis (MOLT-4 cells) depicted in FIG. 16 demonstrated S-phase arrest at 4-8 hours.

Hemolysis assays indicated no RBC lysis at 1.5 μM, suitable for parenteral or systemic administration, corresponding spectrum is shown in FIG. 17.

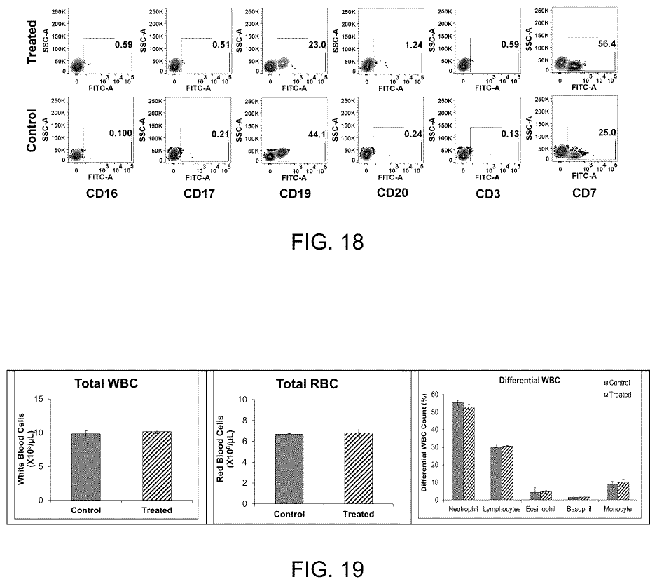

As shown in FIG. 18, ex vivo PBMCs from ALL/AML patients treated with 20 μM curcumin-mercury compound showed increased expression of CD3, CD7, CD19, and CD20, indicating immune-stimulatory activity.

In vivo sub-acute toxicity studies in Swiss-albino mice is performed with intravenous injection and results are shown in FIG. 19, data sets demonstrated no adverse hematological effects; instead, modest increases in RBC and WBC counts were observed.

### Generalization Sentence:

This Example illustrates one species of the organometallic curcumin compounds of claims 1-7, 15 and 17; analogous compounds may be prepared using any metal M listed in claim 1 under similar conditions.

### Example 2: Curcumin-Manganese Compound

### ((1E,6E)-3,5-diketo/dihydroxy-1,7-bis(3-hydroxy-4-methoxyphenyl) hepta-1,6-dien-4-yl) manganese

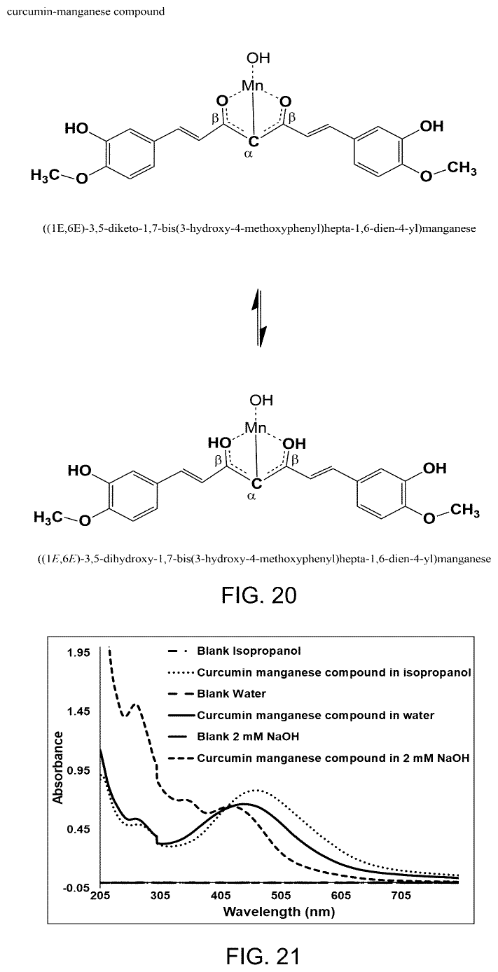

Another species of organometallic compounds of curcumin as claimed in claim 1 and represented in FIG. 1 (formula I) and FIG. 20 is synthesized as follows. Curcumin was dissolved in anhydrous ethanol; water was added to obtain 1 mM curcumin concentration and water content ˜5% (v/v). Ammonium hydroxide was added to adjust pH to 7.5-8.5, followed by addition of manganese chloride (MnCl₂) at a 1:10 molar ratio. The mixture was stirred for 4-5 hours at room temperature (˜25° C.), developing turbidity.

The mixture was centrifuged, and pellets were washed repeatedly with ethanol and water. Pellets were dried and later dissolved in aqueous 4 mM NaOH for analysis. Because the compound dissolves readily in 4 mM NaOH, it is inherently soluble in stronger alkaline media, including 10 mM NaOH, consistent with the solubility characteristics described elsewhere herein.

### Experimental Characterization and Results

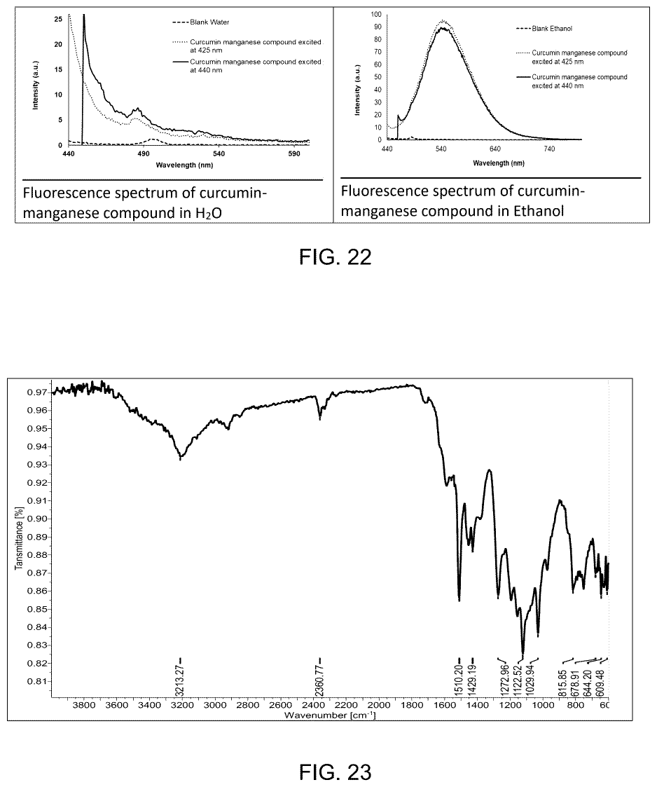

The compound formed bright orange solutions in NaOH. FIG. 21 showing UV-Vis spectra demonstrated λₘₐₓ at 457 nm (isopropanol), 440 nm (water), and 415 nm (NaOH). FIG. 22 showed, fluorescence was negligible in water.

FIG. 23 exhibited FTIR spectra, peaks near 3250 cm⁻¹ (OH), 1650 cm⁻¹ (C═O), and 974 cm⁻¹ consistent with metal coordination.

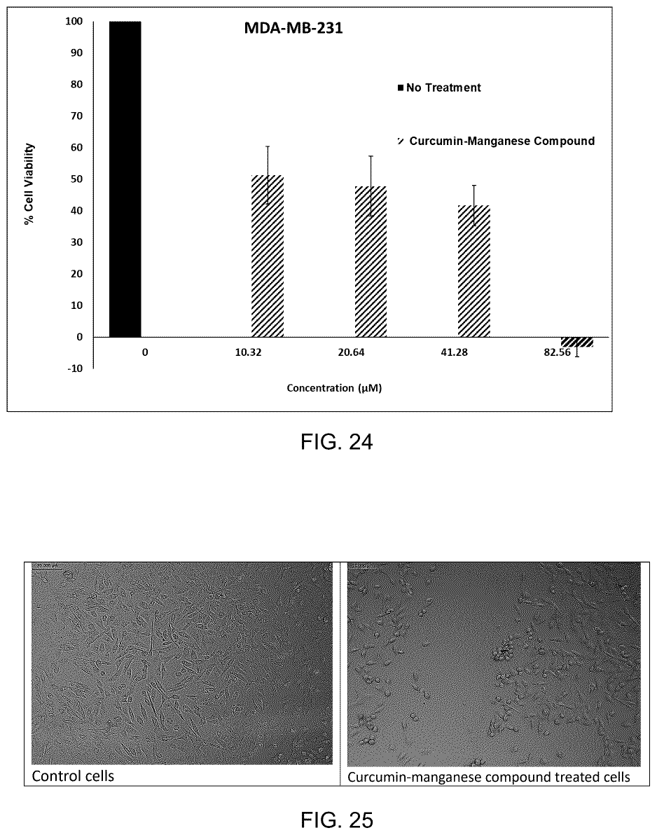

FIG. 24 showed MTT analysis using MDA-MB-231 cells, demonstrated IC₅₀≈10.3 μM. Scratch assays demonstrated inhibition of cell migration, corresponding micrographs are depicted in FIG. 25.

### Generalization Sentence:

This Example demonstrates preparation of a curcumin-metal compound within the scope of claims 1-7, 15, 17; related compounds can be produced using any metal M recited therein.

### Example 3:2-Mercaptophenol-Mercury Complex

### IUPAC Name: [hydroxy ((2-hydroxyphenyl)thio) mercury]

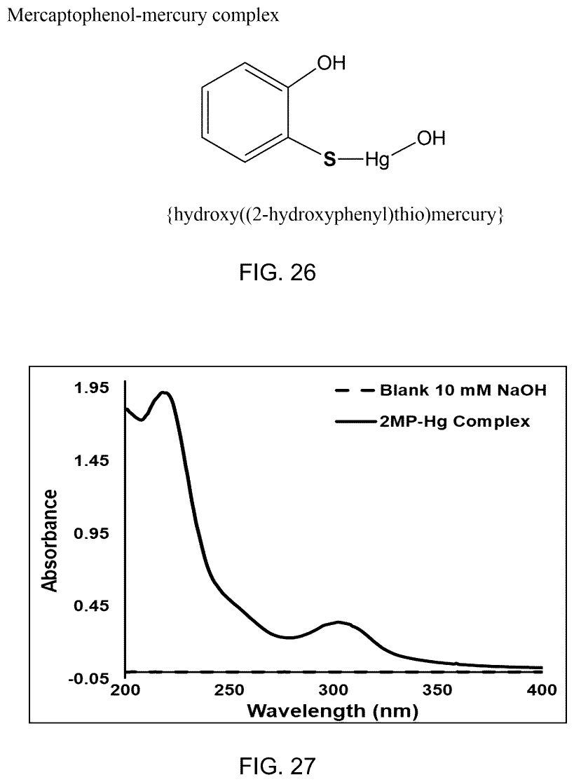

Organometallic complex of mercaptophenol as claimed in claim 8 and represented in FIG. 2 (formula II) and FIG. 26 is synthesized as follows. 2-mercaptophenol was dissolved in anhydrous ethanol, followed by addition of water. Sodium hydroxide was added, followed by mercuric chloride at a 1:1 molar ratio. The reaction mixture turned greenish-yellow. The mixture was chilled and lyophilized to yield solid material for characterization and biological evaluation.

### Experimental Characterization and Results

As depicted in FIG. 27, UV-Vis spectroscopy (10 mM NaOH) showed a bathochromic shift of the 293-nm peak to 302 nm and a hypsochromic shift of the 255 nm band to 216 nm.

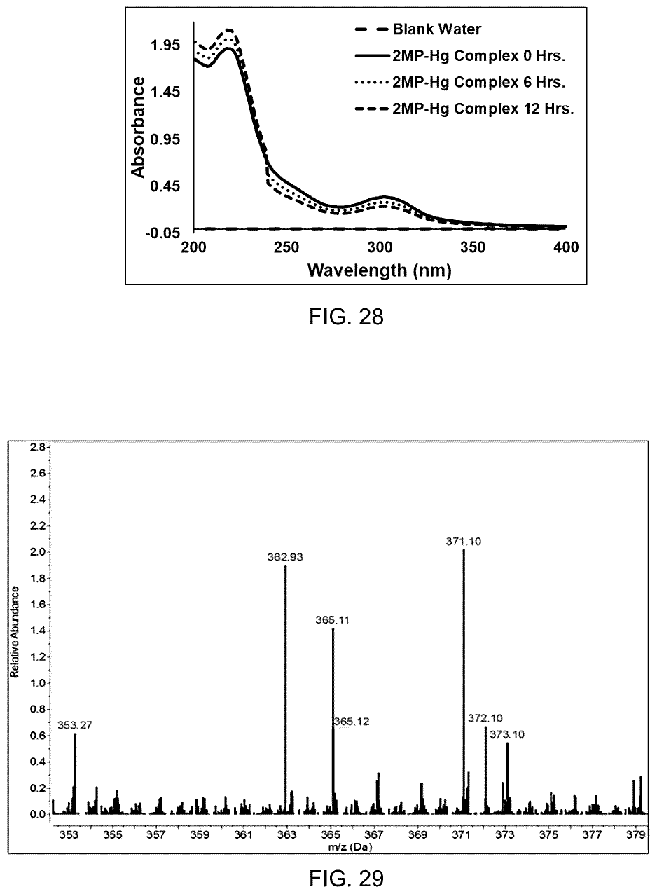

As shown in FIG. 28, overall pattern of the UV-Vis spectra remained unchanged over 12 hours at room temperature, indicating stability.

ESI-MS showed a peak at m/z 365.11, matching the expected mass as sodium adduct, corresponding data set is presented in FIG. 29.

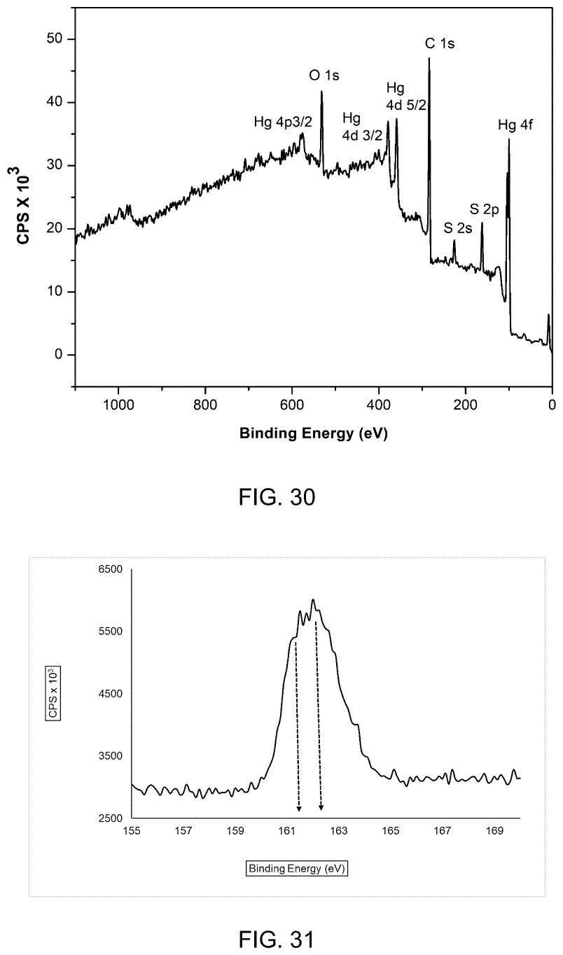

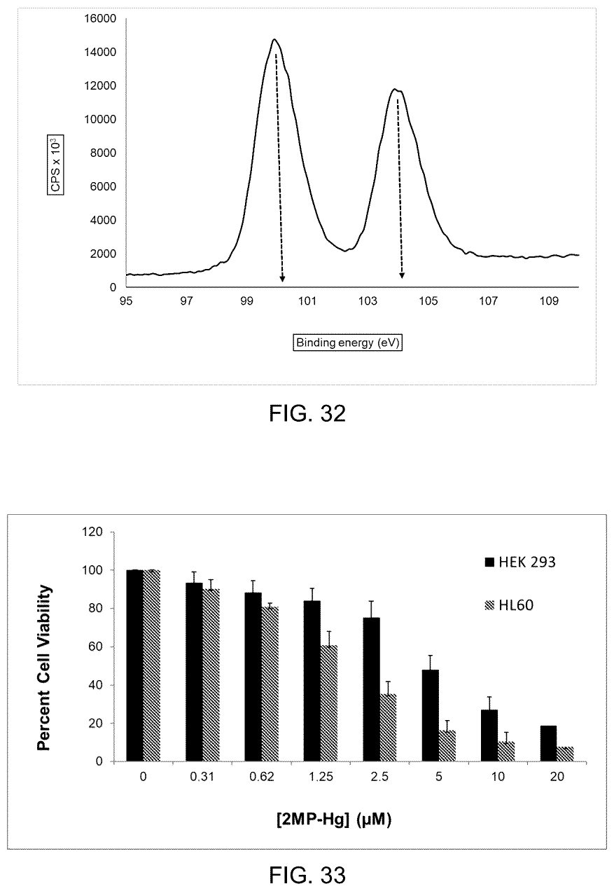

As shown in FIG. 30, XPS confirmed presence of all elements as expected for 2-mercaptophenol-mercury complex. FIG. 31 further showed a broad peak covering the region of 159.8-165 eV indicate presence of sulfur in metal bonded form. Protruding shoulder at 161.5 eV specially indicates the binding of mercury with the sulfur atom, while the maxima around 162.2 eV indicates the sulfur bonding with carbon. Due to very close proximity of binding energies of the said forms of sulfur (—S—Hg, —S—C, —SH) they cannot be deconvoluted out from the acquired spectrum, as shown in FIG. 31. Furthermore, as shown in FIG. 32, two intensified peaks of mercury (Hg 4f 7/2 and Hg 4f 5/2) at 100.3 eV and 104 eV, derived from XPS spectrum reconfirmed the bonding of mercury atom with the sulfur atom of 2-mercaptophenol.

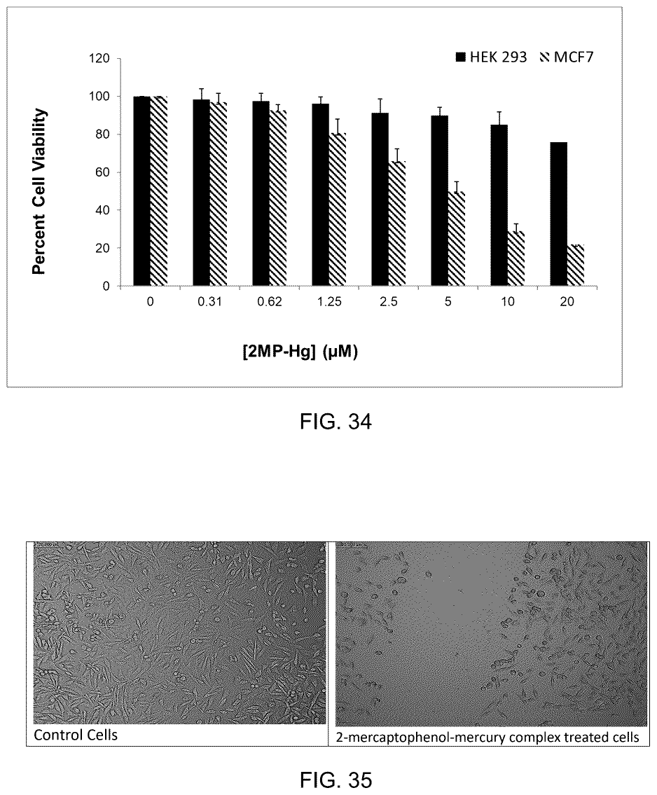

As shown in FIGS. 33 and 34, MTT assays demonstrated strong antiproliferative activity against HL-60 and MCF7 cells, with IC₅₀ values of ˜2.0 μM and ˜7.5 μM, respectively, while little cytotoxicity is observed against non-cancerous cells HEK-293.

Scratch assays showed inhibition of MDA-MB-231 cell migration, corresponding micrographs are displayed in FIG. 35.

All in vitro as well as ex vivo cells were cultured under standard conditions, and candidate complex was filtered before use.

### Generalization Sentence:

This Example illustrates one species of the mercaptophenol-metal complexes described in claims 8-14, 16 and 18; analogous complexes may be prepared using any metal or nonmetal atom X and mercaptophenol isomer recited therein.

### Example 4:2-Mercaptophenol-Thio-Manganese Complex (2MP-Thio-Manganese Complex)

### IUPAC Name: [bis((2-hydroxyphenyl)thio) manganese]

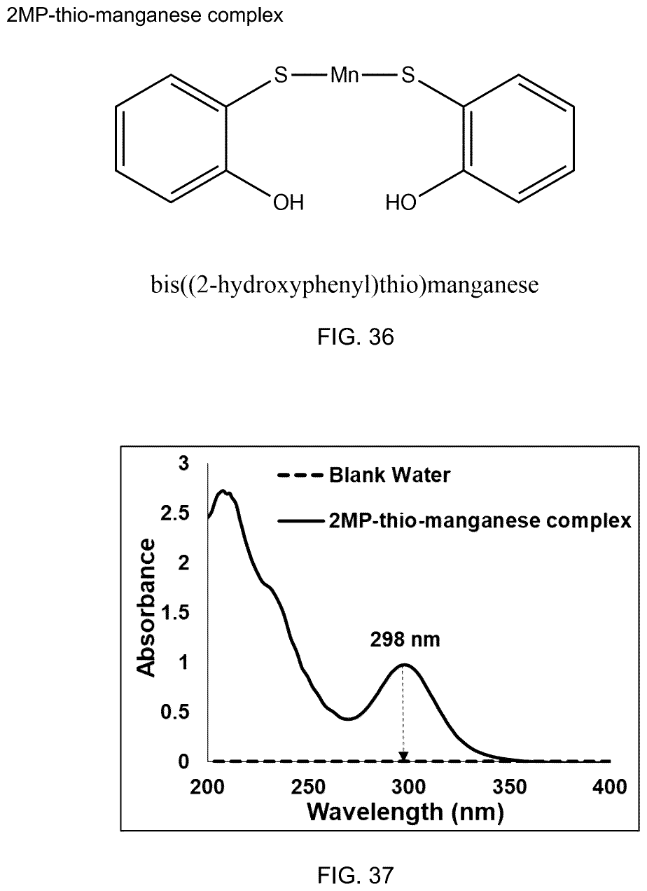

Organometallic complex of mercaptophenol as claimed in claim 8 and represented in FIG. 2 (formula II) and FIG. 36 is synthesized as follows. 2-mercaptophenol was dissolved in anhydrous ethanol, followed by addition of solution of manganese chloride at a molar ratio of 1:5 (2-mercaptophenol:Manganese=1:5). Then, aqueous solution of ammonium hydroxide was added at a molar ratio of 1:2.5 (2-mercaptophenol:NH₄OH=1:2.5). The reaction mixture turned yellow and incubated for around 30 min at room temperature (18-25° C.), followed by drying under vacuum, then subsequently washing with n-hexane, chloroform, ethanol and collection of respective soluble fractions. Chloroform and ethanol fractions were further dried under vacuum and resuspended in pure water with sonication and centrifugation and supernatant was used for characterization and biological evaluation.

### Experimental Characterization and Results

As depicted in FIG. 37, UV-Vis spectroscopy in pure water showed an absorbance band around 298 nm.

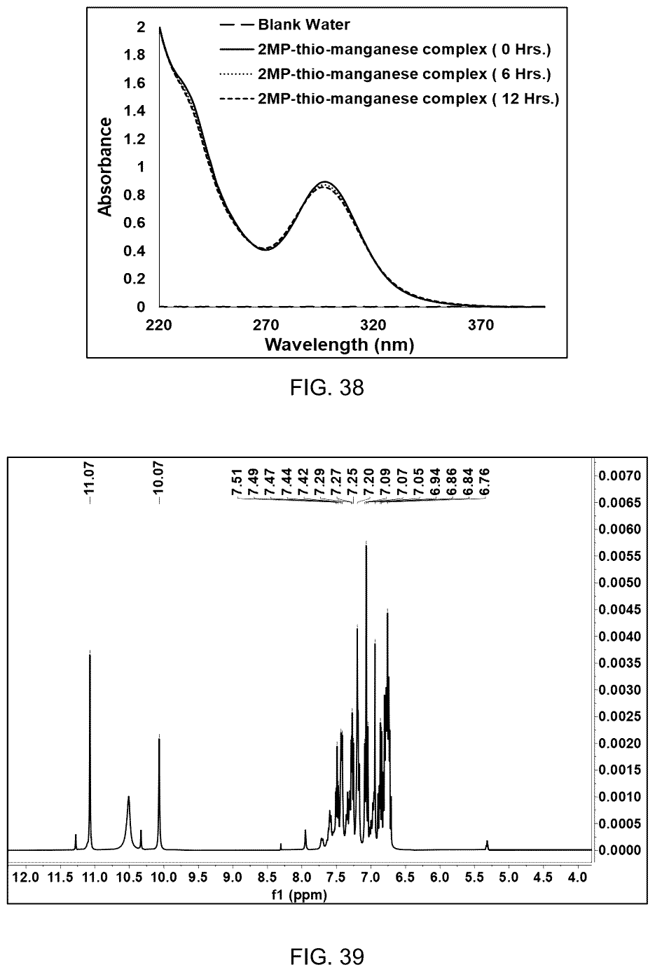

As shown in FIG. 38, UV-Vis spectra almost remained unchanged over 12 hours at room temperature, retaining ≥95% of the absorbance at its λₘₐₓ, 298 nm.

¹H NMR as shown in FIG. 39 demonstrated loss of the sulfur-associated proton signal at 4.53 ppm, confirming substitution at thiol sulfur. Conjugated peaks of phenolic ring protons is observed at 7.51-6.76 ppm, upon complex formation. Peaks in the region of 10-11 ppm confirms the presence of phenolic OH in the complex, without any substitution or interaction with the metal center.

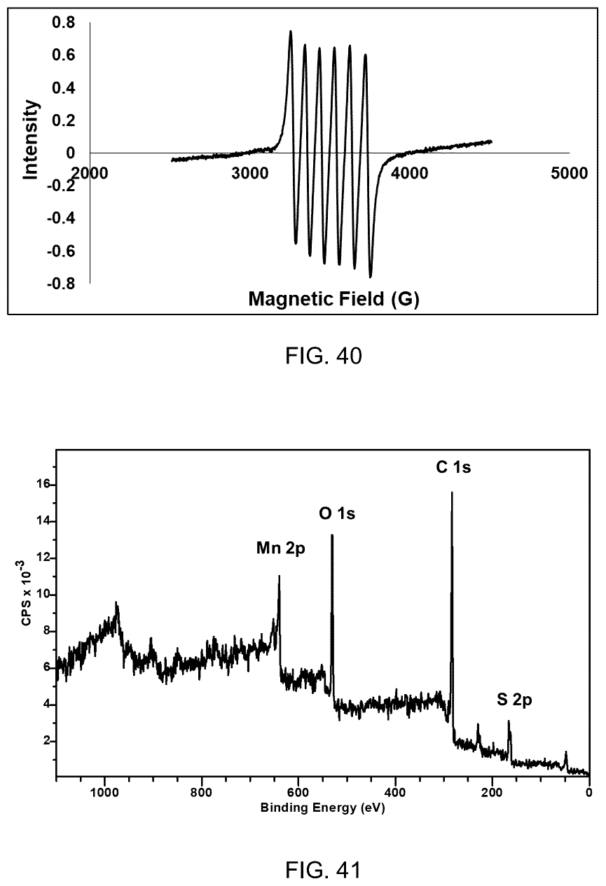

EPR spectrum as shown in FIG. 40 demonstrated well resolved hyperfine sextet in the range of 3200-3800 G, confirming the presence of predominant Mn(II) complex.

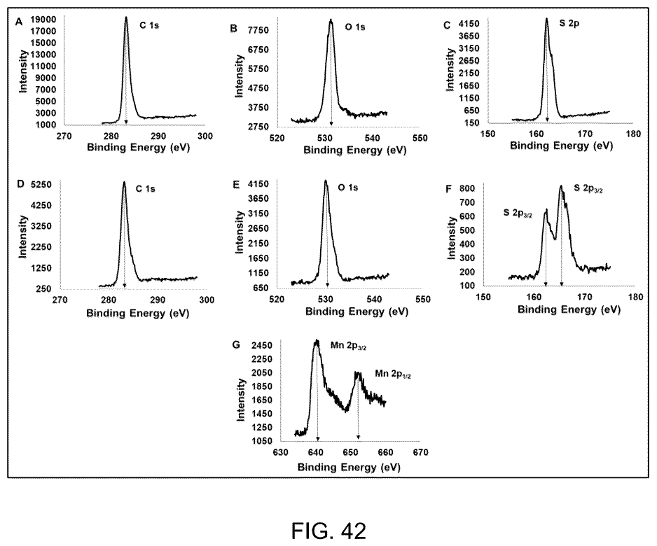

As shown in FIG. 41, XPS spectrum confirmed presence of all elements as expected for 2MP-thio-manganese complex. FIG. 42 (G) showed bands of Mn 2p 3/2 and 2p 1/2 at 640.25 and 652.25 eV, respectively confirming the presence of S—Mn bond in the complex, that is further confirmed by the S 2p band at 162.5 and 165.375 eV. C 1s, O 1s and S 2p XPS spectrum of 2MP are displayed in A, B and C, respectively, while that of 2MP-thio-manganese complex are presented in D, E, F. S 2p in 2MP showed a monomodal distribution, while S 2p in 2MP-thio-manganese complex showed a bimodal distribution, confirming the sulfur coordination.

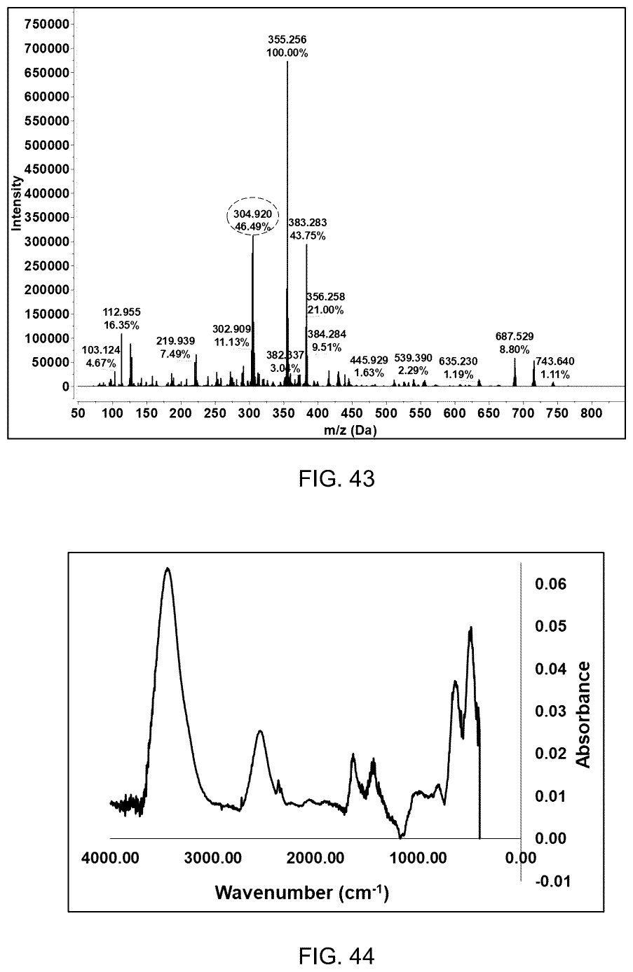

ESI-MS showed a peak at m/z 304.92, matching the expected mass, corresponding data set is presented in FIG. 43.

FTIR spectra in D₂O showed absorbance band in the region of 472.00-511.05 cm⁻¹ matching the expected band for S—Mn stretching and bending vibration. Another band in the region of 615.67-654.24 cm⁻¹ is observed representing the C—S—Mn deformation modes. Corresponding data set is presented in FIG. 44 confirming the sulfur coordination.

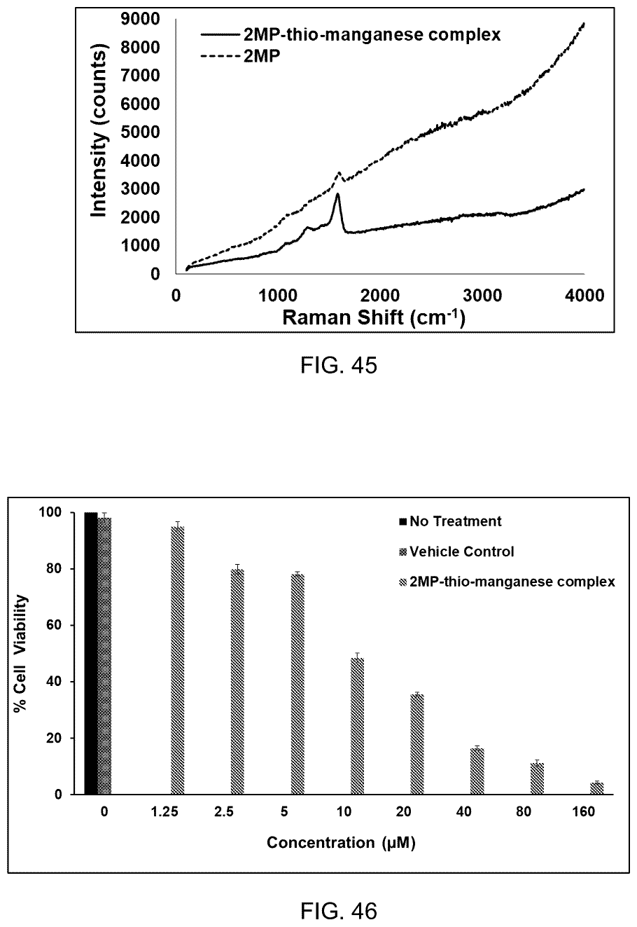

As shown in FIG. 45, Raman spectra showed a band shift from 1596.96 cm⁻¹ to 1583.4 cm⁻¹ upon 2MP-thio-maganese complex formation; further a significant quenching of background fluorescence, along with reduction in peak intensity (1583.4 cm⁻¹) and peak broadening are also observed in 2MP-thio-manganese complex, comparing to 2-mercaptophenol.

As shown in FIG. 46, MTT assays demonstrated strong antiproliferative activity against MDA-MB-231 cells, with IC₅₀ values of ˜10.0 μM.

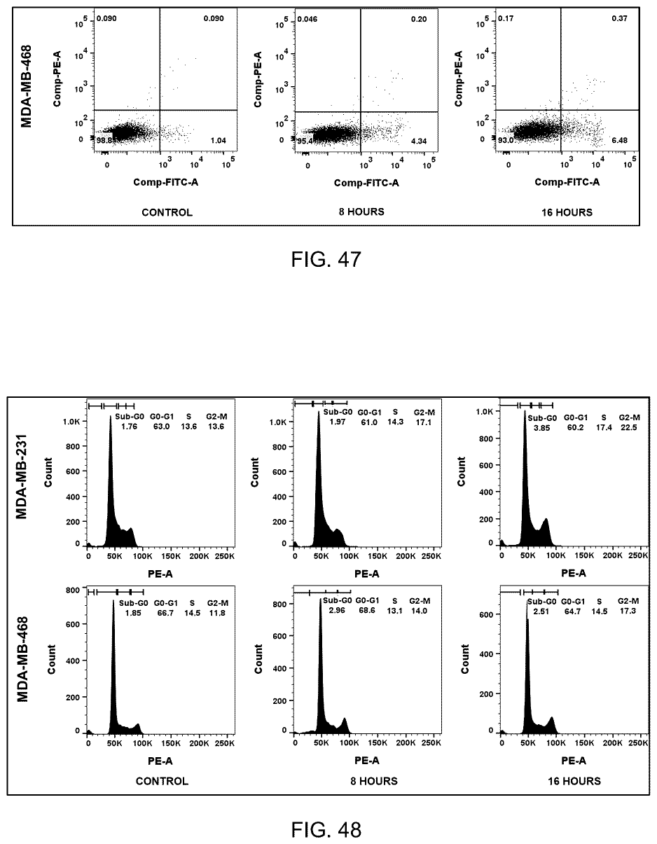

As shown in FIG. 47, Annexin-V/PI staining (MDA-MB-468 cells) showed time-dependent apoptosis without necrosis upon 10 μM 2MP-thio-manganese complex treatment. Pl-stained cell-cycle analysis of MDA-MB-231 and MDA-MB-468 cells upon 10 μM 2MP-thio-manganese complex treatment demonstrated G2/M-phase arrest at 8-16 hours as depicted in FIG. 48.

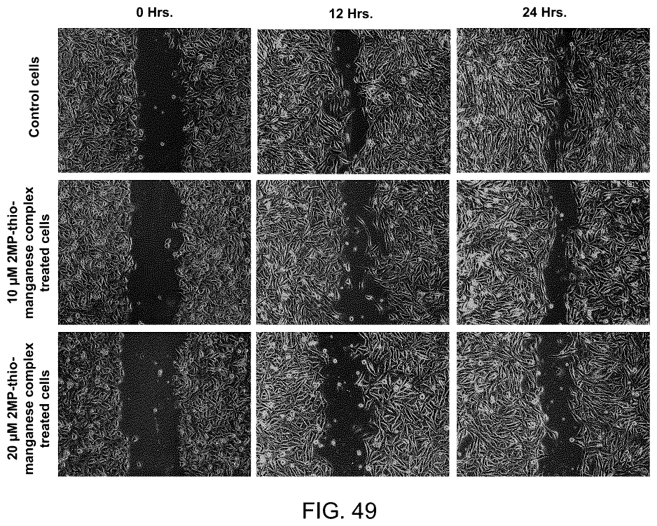

Scratch assays showed inhibition of MDA-MB-231 cell migration upon 10 and 20 μM 2MP-thio-manganese complex treatment, corresponding micrographs are displayed in FIG. 49.

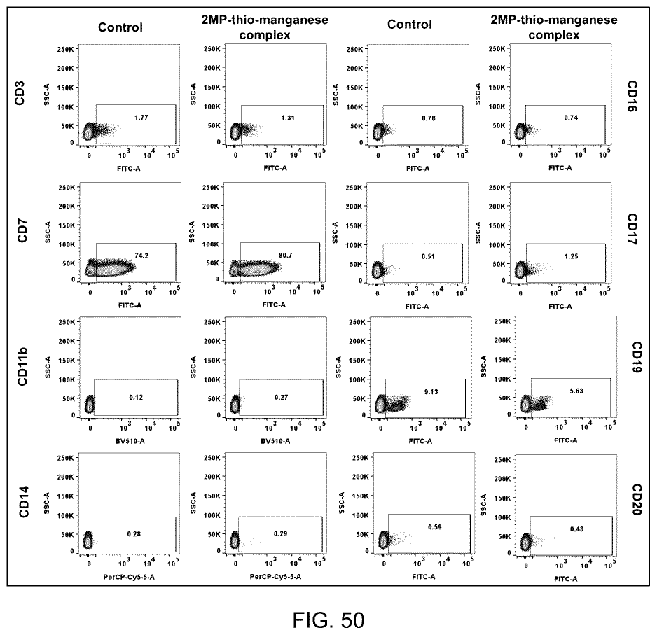

As shown in FIG. 50, ex vivo WBCs from healthy individual treated with 10 μM 2MP-thio-manganese complex demonstrated increased expression of CD7, CD11b, and CD17 confirming immune-stimulatory activity.

All in vitro as well as ex vivo cells were cultured under standard conditions, and candidate complex was filtered before use.

### Generalization Sentence:

This Example illustrates one species of the mercaptophenol-metal complexes described in claims 8-14, 16 and 18; analogous complexes may be prepared using any metal or nonmetal atom X and mercaptophenol isomer recited therein.

### Example 5:2-Mercaptophenol-Selenium Complex (2MP-Selenium Complex)

### IUPAC Name: [S,S-bis(2-hydroxyphenyl) selenodithioite]

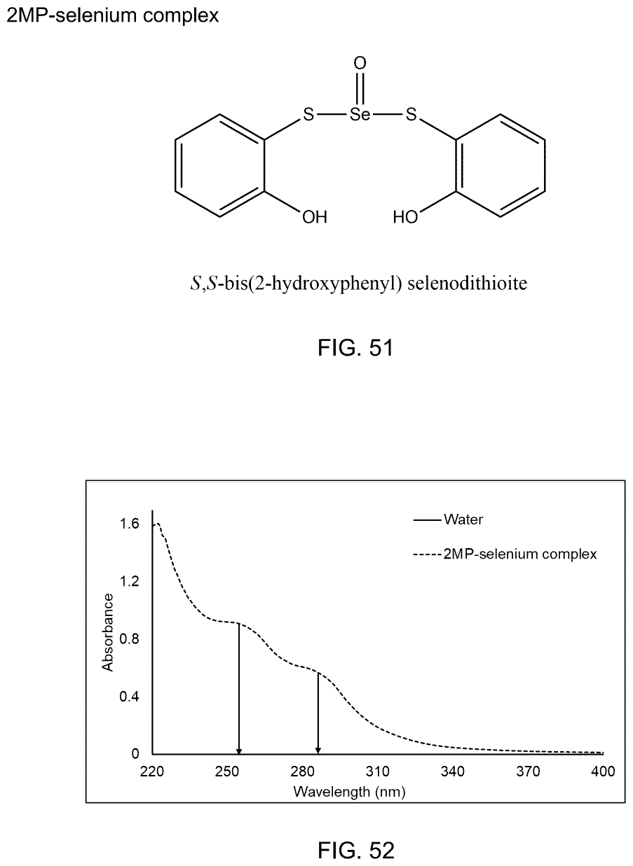

Organometallic complex of mercaptophenol as claimed in claim 8 and represented in FIG. 2 (formula II) and FIG. 51 is synthesized as follows. 2-mercaptophenol stock solution was prepared in acetonitrile, and solution/suspension of selenium chloride is also prepared separately in acetonitrile, followed by addition of the 2-mercapophenol solution to the selenium chloride in a molar ratio of 1:5 (2MP:Selenium=1:5). Then reaction mixture turned yellow and incubated for around 30 min at room temperature (18-25° C.), followed by drying under vacuum, then subsequently washing with n-hexane, dichloromethane, ethanol and collection of respective soluble fractions. Ethanol fraction was further dried under vacuum and resuspended in pure water with sonication and centrifugation and supernatant was used for characterization and biological evaluation.

### Experimental Characterization and Results

As depicted in FIG. 52, UV-Vis spectroscopy in pure water showed two characteristic peaks at 286 and 256 nm.

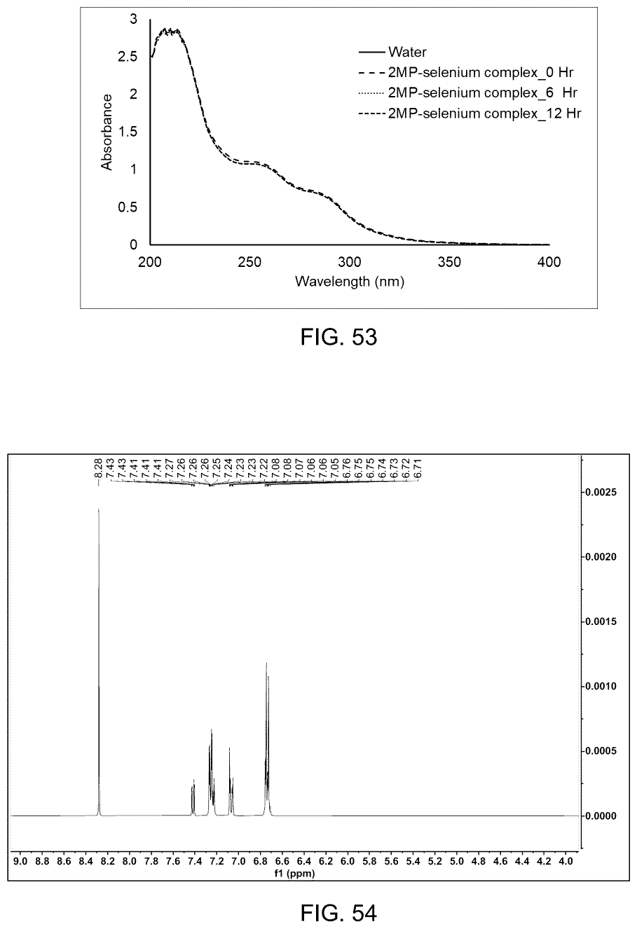

As shown in FIG. 53, UV-Vis spectra almost remained unchanged over 12 hours at room temperature, retaining ≥95% of the absorbance at its λₘₐₓ, 286 and 256 nm.

¹H NMR as shown in FIG. 54 demonstrated loss of the sulfur-associated proton signal at 4.53 ppm, confirming substitution at thiol sulfur. Conjugated peaks of phenolic ring protons is observed at 7.50-6.60 ppm, upon complex formation.

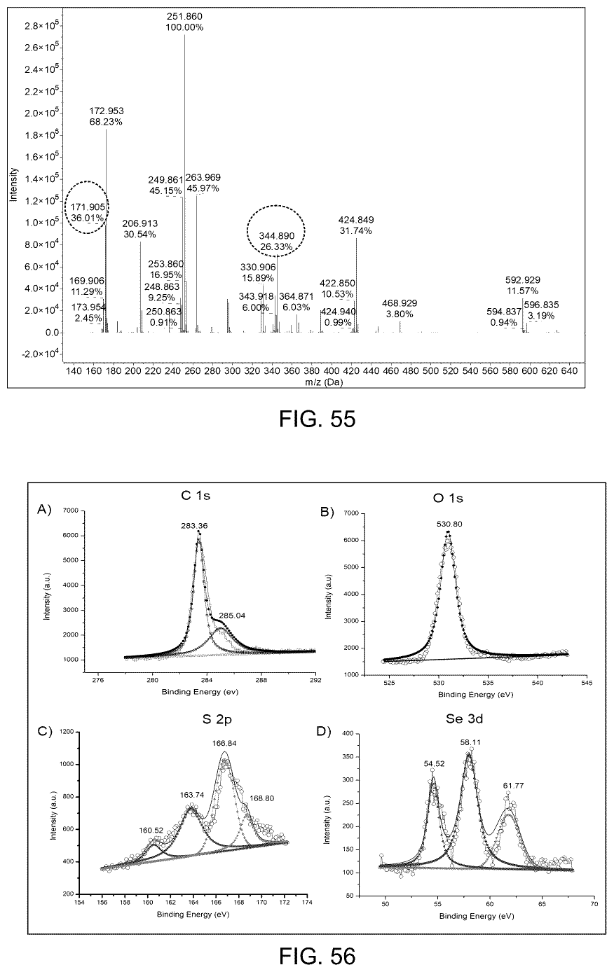

ESI-MS showed a peak at m/z 344.890, matching the expected single de-pronated mass, corresponding data set is presented in FIG. 55.

As shown in FIG. 56, XPS bands of Se 3d at 58.11 eV and 61.77 eV, confirm the presence of S—Se and S—O bond in the complex, that is further confirmed by the S 2p band at 163.74 eV. C 1s, O 1s, S 2p and Se 3d XPS spectrum of 2MP-selenium complex are displayed in A, B, C and D, respectively.

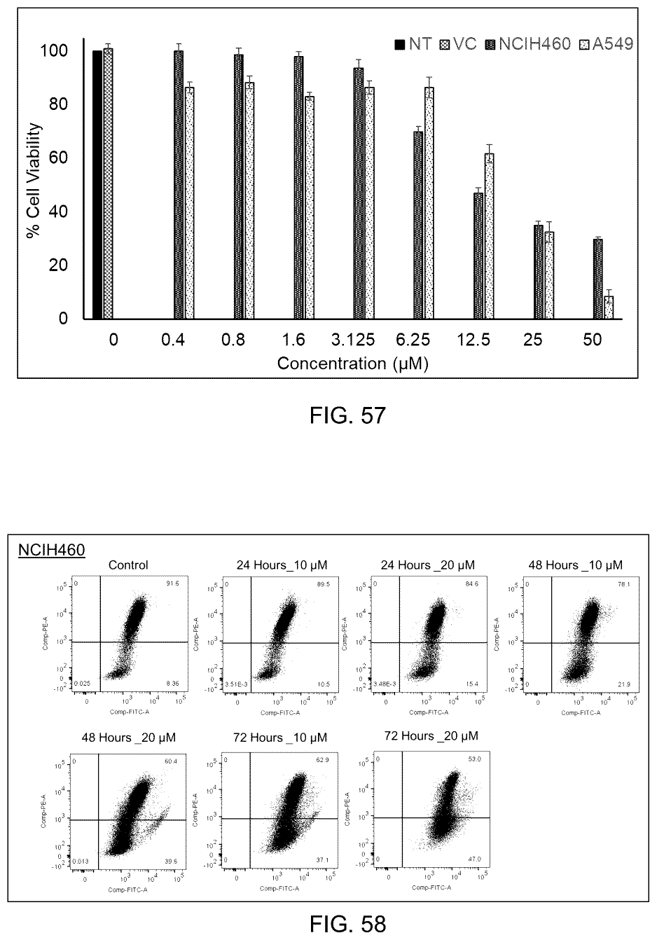

As shown in FIG. 57, MTT assays demonstrated strong antiproliferative activity against A549 and NCIH460 cells, with IC₅₀ values of ˜15.0 and 10.0 μM, respectively.

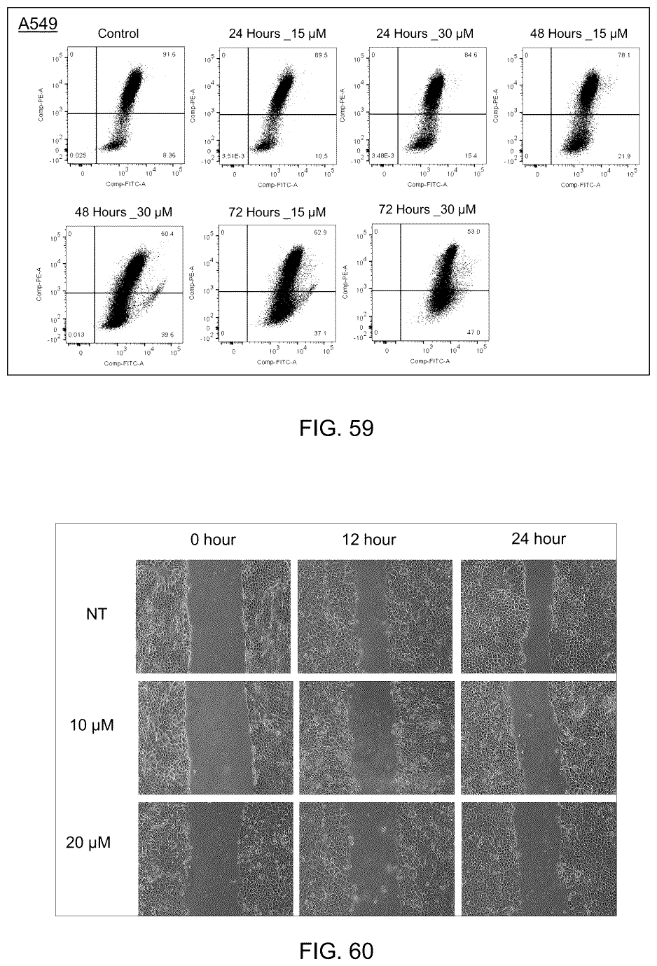

As shown in FIGS. 58 and 59, depolarization of mitochondrial trans-membrane potential of NCIH460 and A549 cells, respectively, with JC1 staining demonstrated induction of apoptosis in a time dependent manner.

Scratch assays showed inhibition of NCIH460 cell migration upon 10 and 20 μM 2MP-selenium complex treatment, corresponding micrographs are displayed in FIG. 60.

All in vitro as well as ex vivo cells were cultured under standard conditions, and candidate complex was filtered before use.

### Generalization Sentence:

This Example illustrates one species of the mercaptophenol-metalloid complexes described in claims 8-14, 16 and 18; analogous complexes may be prepared using any metal or nonmetal atom X and mercaptophenol isomer recited therein.

### Example 6:2-Mercaptophenol-Sulfoxide Dimer (2MP-Sulfoxide Dimer)

### IUPAC Name: [S-(2-hydroxyphenyl) 2-hydroxybenzenesulfinothioate]

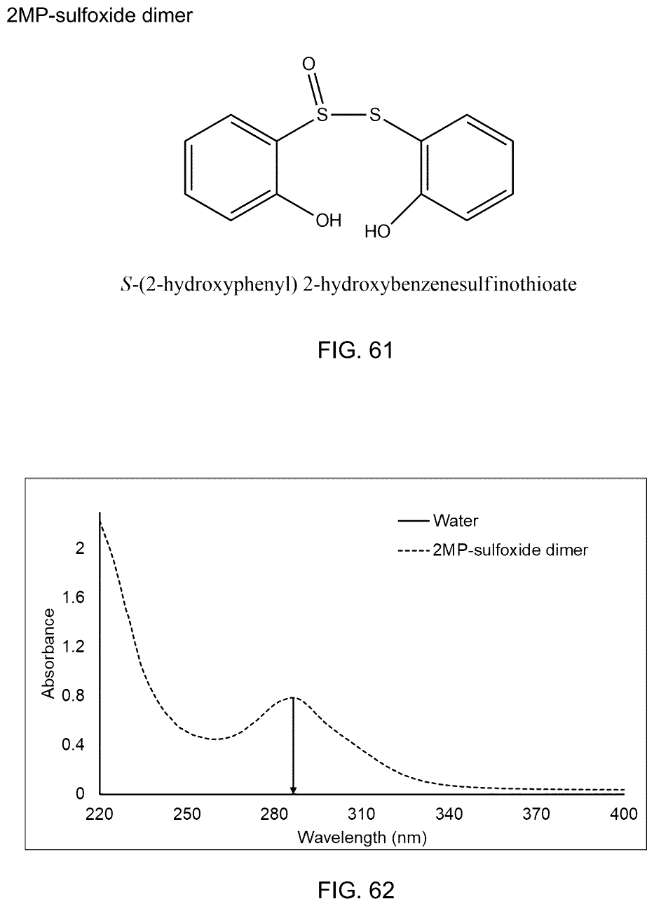

Mercaptophenol derivative as claimed in claim 8 and represented in FIG. 2 (formula II) and FIG. 61 is synthesized as follows. 2-mercaptophenol stock solution was prepared in acetonitrile, and solution/suspension of selenium chloride is also prepared separately in acetonitrile, followed by addition of the 2-mercapophenol solution to the selenium chloride in a molar ratio of 1:5 (2MP:Selenium=1:5). Then reaction mixture turned yellow and incubated for around 30 min at room temperature (18-25° C.), followed by drying under vacuum, then subsequently washing with n-hexane, dichloromethane, ethanol and collection of respective soluble fractions. Dichloromethane fraction was further dried under vacuum and resuspended in pure water with sonication and centrifugation and supernatant was used for characterization and biological evaluation.

### Experimental Characterization and Results

As depicted in FIG. 62, UV-Vis spectroscopy in pure water showed characteristic peak at 286 nm.

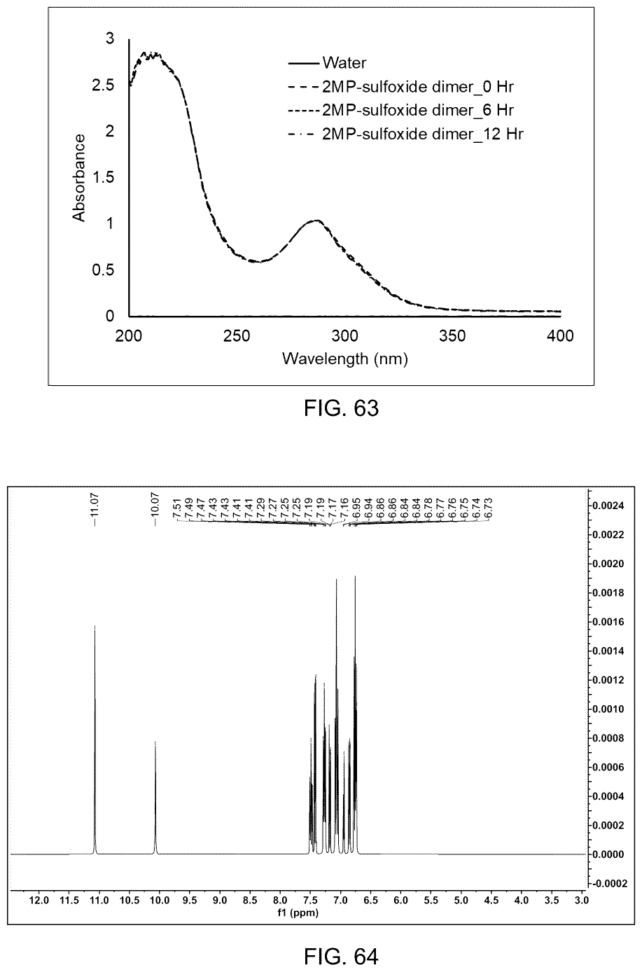

As shown in FIG. 63, UV-Vis spectra almost remain unchanged over 12 hours at room temperature, retaining ≥95% of the absorbance at its λₘₐₓ, 286 nm.

¹H NMR as shown in FIG. 64 demonstrated loss of the sulfur-associated proton signal at 4.53 ppm, confirming substitution at thiol sulfur. Conjugated peaks of phenolic ring protons is observed at 7.70-6.70 ppm, upon derivatization. Peaks in the region of 10-11 ppm confirms the presence of phenolic OH in the 2MP-sulfoxide dimer, without any substitution or interaction of the hydroxyl oxygen.

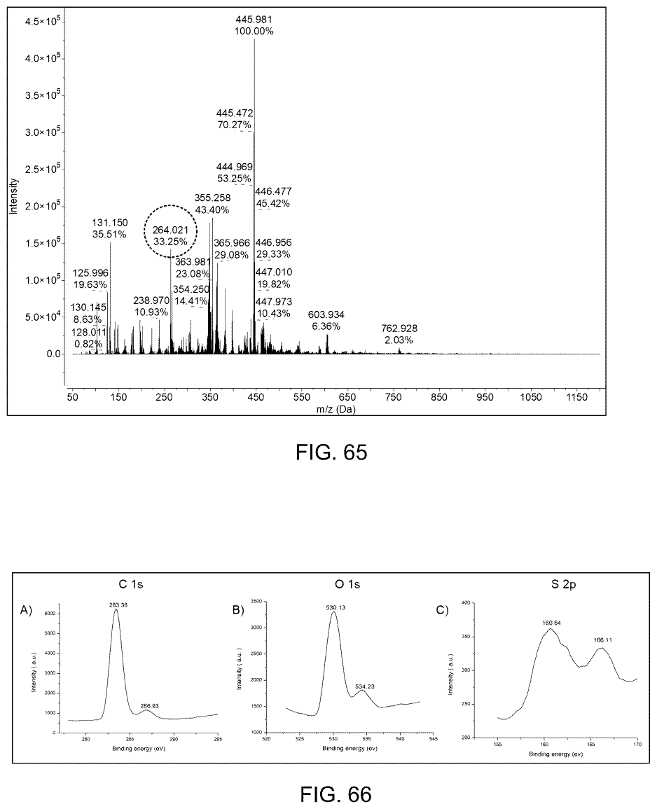

ESI-MS showed a peak at m/z 264.021, matching the expected single de-pronated mass, corresponding data set is presented in FIG. 65.

As shown in FIG. 66, XPS bands of S 2p at 166.11 eV and O 1s at 530.13 eV confirm the presence of S—O bond in the 2MP-sulfoxide dimer. C 1s, O 1s and S 2p XPS spectrum of 2MP-sulfoxide dimer are displayed in A, B and C, respectively.

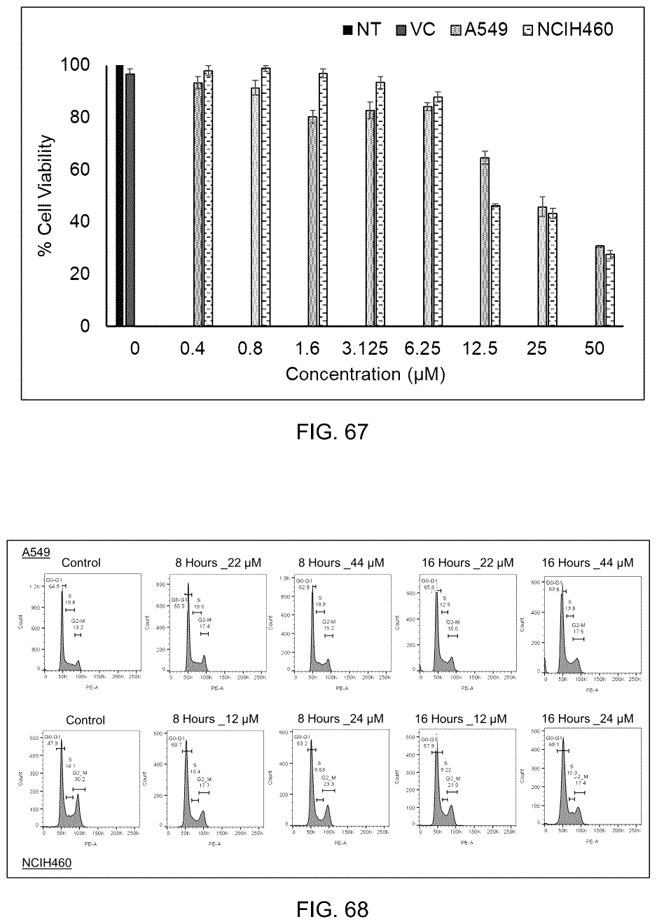

As shown in FIG. 67, MTT assays demonstrated strong antiproliferative activity against A549 and NCIH460 cells, with IC₅₀ values of ˜22.0 and 12.0 μM, respectively.

PI-stained cell-cycle analysis of A549 and NCIH460 cells demonstrated that the 2MP-sulfoxide dimer treatment can arrest cell cycle at G2/M and G0/G1 phase, respectively, in a time dependent manner; corresponding data sets is shown in FIG. 68.

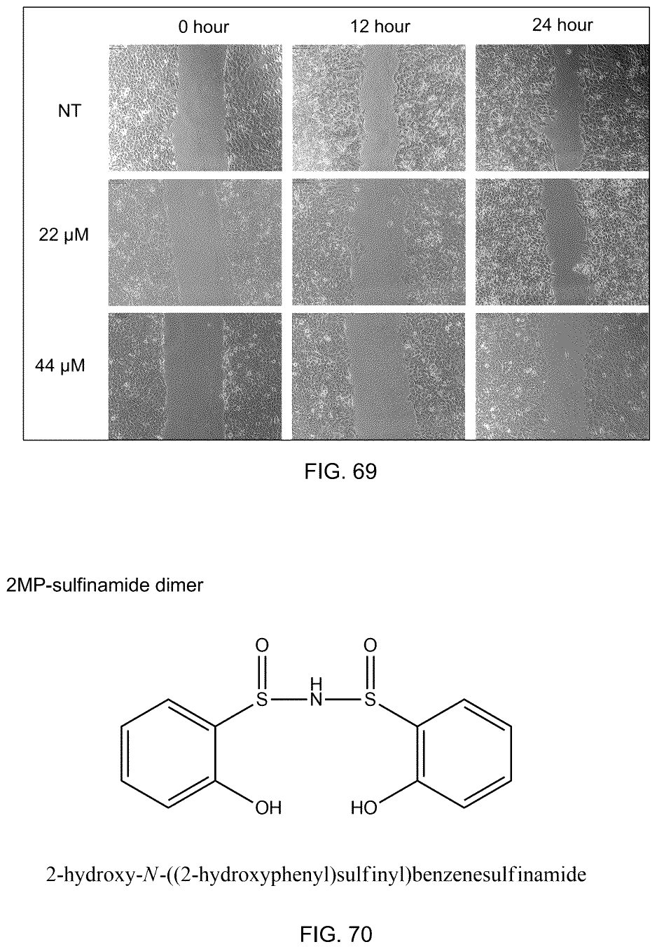

Scratch assays showed inhibition of A549 cell migration upon 22 and 44 μM of 2MP-sulfoxide dimer treatment, corresponding micrographs are displayed in FIG. 69.

All in vitro as well as ex vivo cells were cultured under standard conditions, and candidate derivative was filtered before use.

### Generalization Sentence:

This Example illustrates one species of the mercaptophenol derivative described in claims 8-14, 16 and 18; analogous derivatives may be prepared using any metal or nonmetal atom X and mercaptophenol isomer recited therein.

### Example 7:2-Mercaptophenol-Sulfinamide Dimer (2MP-Sulfinamide Dimer)

### IUPAC Name: [2-hydroxy-N-((2-hydroxyphenyl) sulfinyl)benzenesulfinamide]

Mercaptophenol derivative as claimed in claim 8 and represented in FIG. 2 (formula II) and FIG. 70 is synthesized as follows. 2-mercaptophenol was dissolved in anhydrous ethanol, followed by addition of solution of manganese chloride at a molar ratio of 1:5 (2-mercaptophenol:Manganese=1:5). Then, aqueous solution of ammonium hydroxide was added at a molar ratio of 1:2.5 (2-mercaptophenol:NH₄OH=1:2.5). The reaction mixture turned yellow and incubated for around 30 min at room temperature (18-25° C.), followed by drying under vacuum, then subsequently washing with n-hexane, chloroform, ethanol and collection of respective soluble fractions. n-hexane fractions were further dried under vacuum and resuspended in pure water with sonication and centrifugation and supernatant was used for characterization and biological evaluation.

### Experimental Characterization and Results

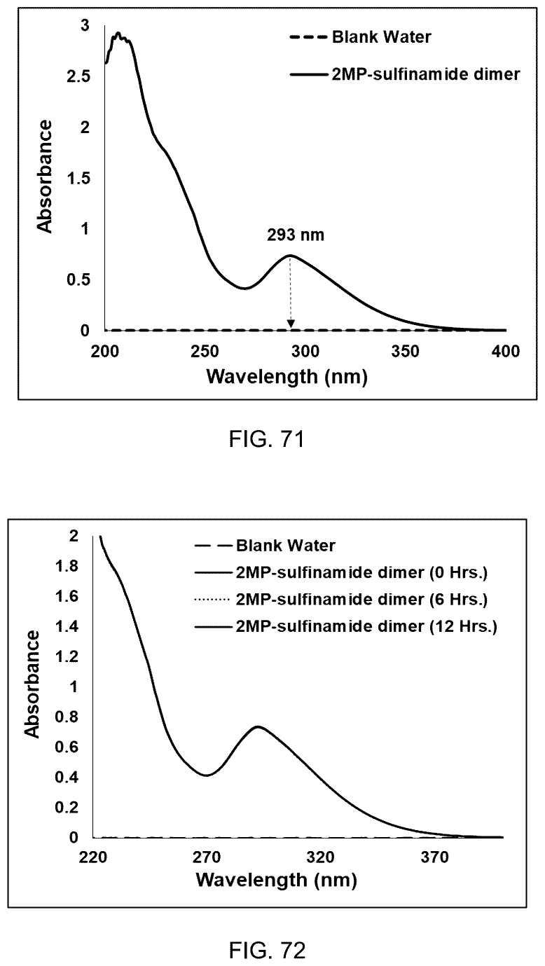

As depicted in FIG. 71, UV-Vis spectroscopy in pure water showed an absorbance band at 293 nm.

As shown in FIG. 72, UV-Vis spectra almost remained unchanged over 12 hours at room temperature, retaining ≥95% of the absorbance at its λₘₐₓ, 293 nm.

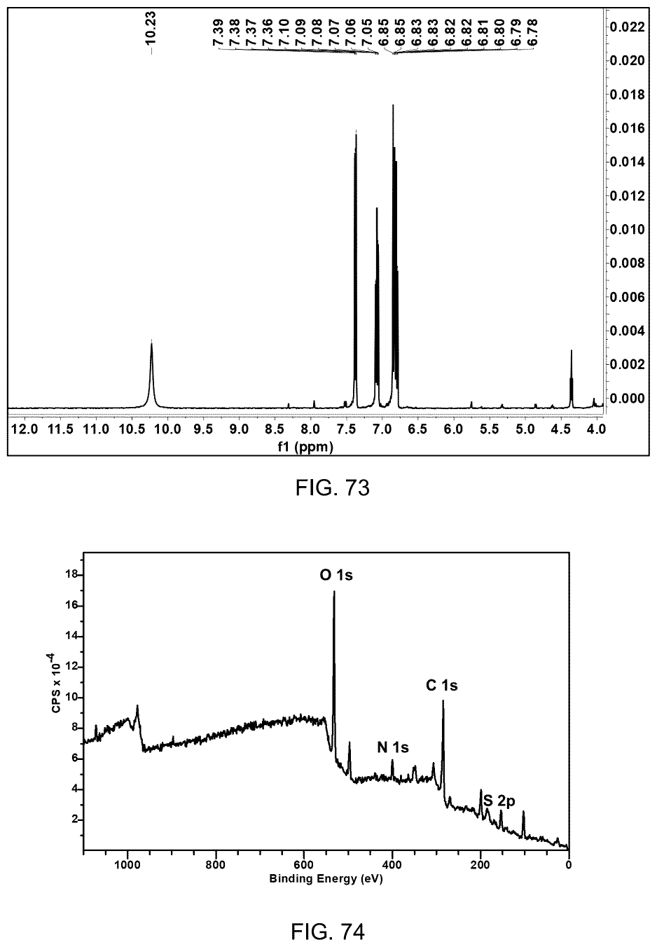

¹H NMR as shown in FIG. 73 demonstrated loss of the sulfur-associated proton signal at 4.53 ppm, confirming substitution at thiol sulfur. Conjugated peaks of phenolic ring protons is observed at 7.39-6.78 ppm, upon dimer formation. Peak at 10.23 ppm confirms the presence of phenolic OH in the dimer, without any substitution or involvement of the hydroxyl oxygen.

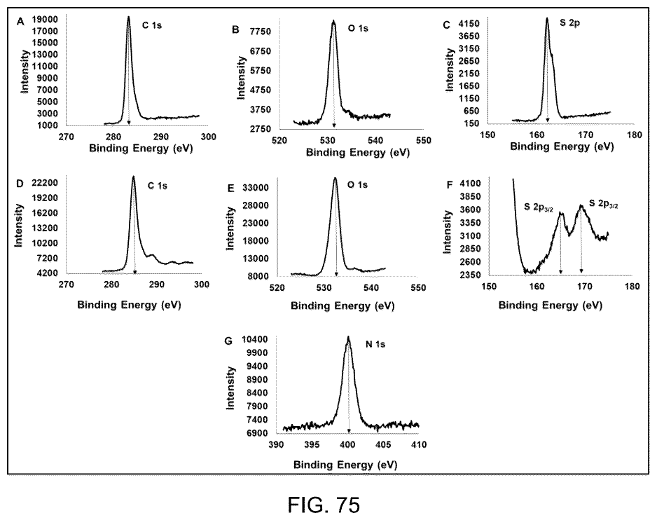

As shown in FIG. 74, XPS spectrum confirmed presence of all elements as expected for 2MP-sulfinamide dimer. FIG. 75 (G) showed bands of N 1s at 400.30 eV confirming the presence of S—N—S bonding, that is further confirmed by the S 2p band at 164.8 eV. Furthermore, S 2p and O 1s bands at 169.1 and 532.3 eV, respectively demonstrate presence of S—O bond in 2MP-sulfinamide dimer. C 1s, O 1s and S 2p XPS spectrum of 2MP are displayed in A, B and C, respectively, while that of 2MP-sulfinamide dimer are presented in D, E, F. S 2p in 2MP showed a monomodal distribution, while S 2p in 2MP-sulfinamide dimer showed a bimodal distribution, confirming the sulfur coordination.

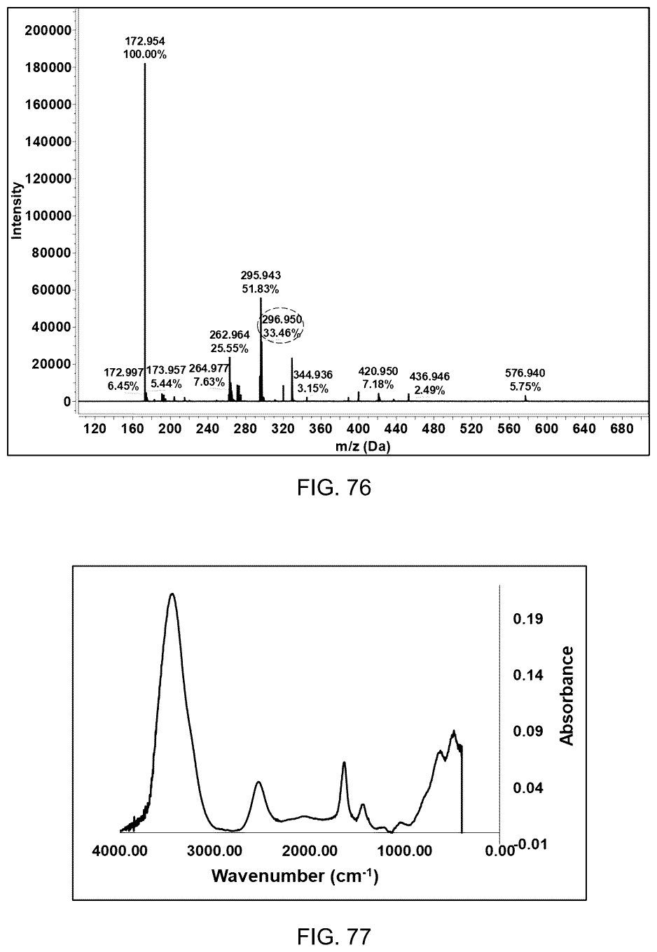

ESI-MS showed a peak at m/z 296.95, matching the expected mass, corresponding data set is presented in FIG. 76.

FTIR spectra in D₂O showed absorbance band in the region of 467.18-508.16 cm⁻¹ matching the expected band for S—N—S bending vibration. Another band in the region of 578.55-667.26 cm⁻¹ is observed representing the S—N stretching and S—O bending vibrations. Further the band in the region of 3418.74-3484.31 cm⁻¹ represents N—H stretching vibration of the sulfinamide. Corresponding data set is presented in FIG. 77 confirm the sulfur coordination.

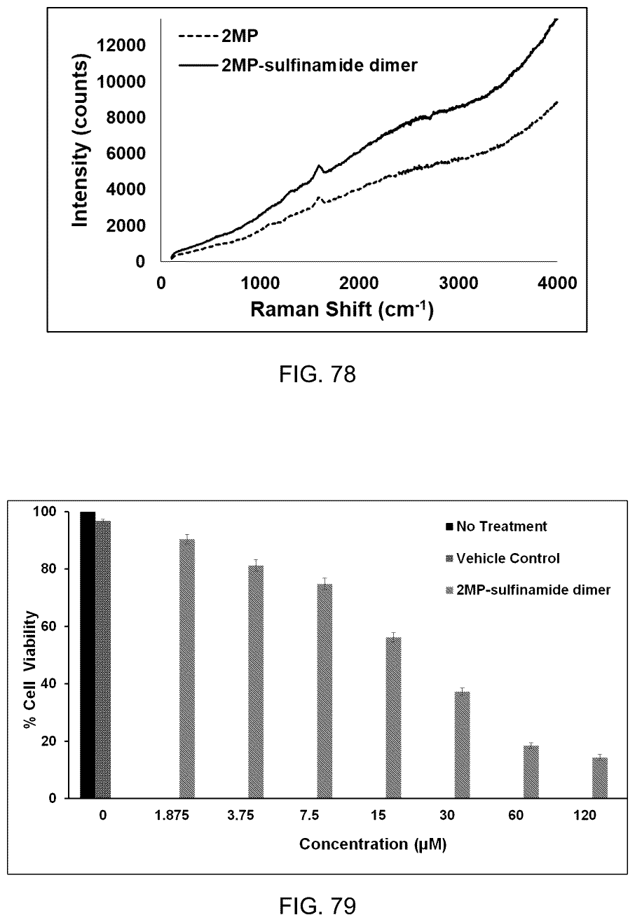

As shown in FIG. 78, Raman spectra showed a band shift from 1596.96 cm⁻¹ to 1587.92 cm⁻¹ upon 2MP-sulfinamide dimer formation; further a significant enhancement of background fluorescence is also observed.

As shown in FIG. 79, MTT assays demonstrated strong antiproliferative activity against MDA-MB-468 cells, with IC₅₀ values of ˜15.0 μM.

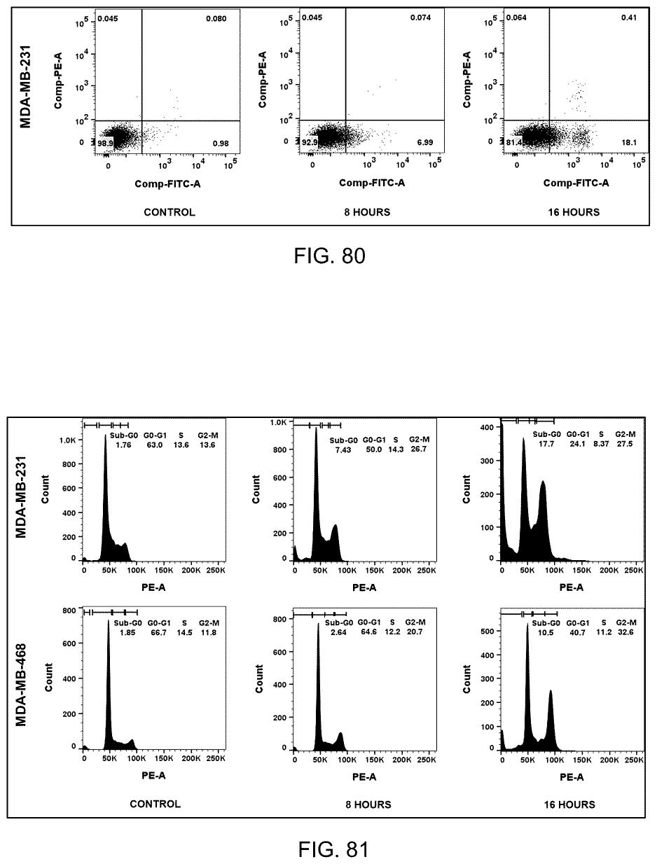

As shown in FIG. 80, Annexin-V/PI staining (MDA-MB-231 cells) showed time-dependent apoptosis without necrosis, upon 15 μM 2MP-sulfinamide dimer treatment. PI-stained cell-cycle analysis of MDA-MB-231 and MDA-MB-468 cells upon 15 μM 2MP-sulfinamide dimer treatment demonstrated G2/M-phase arrest at 8-16 hours as depicted in FIG. 81.

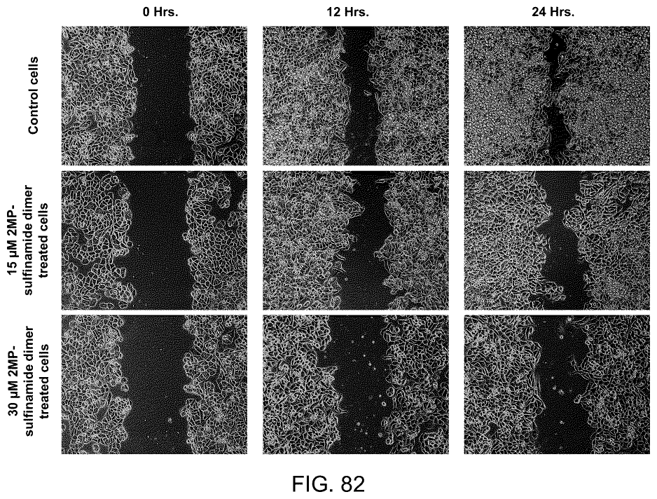

Scratch assays showed inhibition of MDA-MB-468 cell migration upon 15 and 30 μM 2MP-sulfinamide dimer treatment, corresponding micrographs are displayed in FIG. 82.

As shown in FIG. 83, ex vivo WBCs from healthy individual treated with 15 μM 2MP-sulfinamide dimer demonstrated increased expression of CD7, CD11b, CD14 and CD16 confirming immune-stimulatory activity.

All in vitro as well as ex vivo cells were cultured under standard conditions, and candidate complex was filtered before use.

### Generalization Sentence:

This Example illustrates one species of the mercaptophenol derivative described in claims 8-14, 16 and 18; analogous derivatives may be prepared using any metal or nonmetal atom X and mercaptophenol isomer recited therein.

1. An organometallic compound of a curcumin-based molecule having an sp²-hybridized conjugated system, represented by formula I, wherein an α-carbon atom located between two consecutive ionizable keto/enol groups is bonded to a metal or metalloid M;
wherein M is selected from vanadium (V), manganese (Mn), cobalt (Co), nickel (Ni), copper (Cu), zinc (Zn), gallium (Ga), germanium (Ge), arsenic (As), selenium (Se), molybdenum (Mo), silver (Ag), cadmium (Cd), antimony (Sb), platinum (Pt), gold (Au), mercury (Hg), thallium (TI), lead (Pb), and bismuth (Bi);
and wherein one or more remaining coordination sites of M are occupied by ligands R selected from H, —OH, H₂O, —COO—, CO₃—, NO₃—, SO₄—, PO₄—, —SH, —NH₃, —NH₂, —NH, Cl, Br, and I.

2. The compound of claim 1, having an aqueous solubility of at least 2 mM at 18-25° C., when formulated as a sodium, potassium, or calcium salt at a pH of about 7.2 to 10.0, and remaining stable in such aqueous medium for at least 12 hours.

3. The compound of claim 1, capable of forming a non-covalent conjugate or complex with an amphipathic molecule selected from a nucleotide, an oligonucleotide, a chemotherapeutic nucleoside, a chemotherapeutic nucleotide, a chemotherapeutic oligonucleotide, or polyethylene glycol.

4. The compound of claim 1, wherein the curcumin-based molecule comprises two ionizable keto/enol groups in conjugation, and is selected from a curcumin, a curcumin derivative, or a curcumin precursor having two ionizable keto/enol groups in conjugation.

5. The compound of claim 1, wherein the α-carbon exhibits partial double-bond character with adjacent β-carbon atoms.

6. The compound of claim 1, exhibiting antiproliferative, anti-migratory, and anti-invasive activity at a concentration of about 0.5-25 μM against malignant or disease-causing cells.

7. The compound of claim 1, exhibiting immune-modulatory activity, comprising enhancement of blood-cell production or modulation of expression of immune-cell surface markers or receptors at concentration of about 0.5-25 μM.

8. A complex and derivative comprising a mercaptophenol having an sp²-hybridized or conjugated system, represented by Formula II,
wherein the sulfur atom (—S—) of a thiol group (—SH) of at least one mercaptophenol unit is bonded exclusively to an atom X without concurrent bonding to the hydroxyl oxygen of the same mercaptophenol unit;
wherein X is selected from:
(i) a metal or metalloid selected from vanadium (V), manganese (Mn), cobalt (Co), nickel (Ni), copper (Cu), zinc (Zn), gallium (Ga), germanium (Ge), arsenic (As), selenium (Se), molybdenum (Mo), silver (Ag), cadmium (Cd), antimony (Sb), platinum (Pt), gold (Au), mercury (Hg), thallium (TI), lead (Pb), and bismuth (Bi); and
(ii) a non-metal selected from oxygen (O), nitrogen (N), and sulfur(S);
and wherein, when X is a metal or metalloid, any remaining coordination sites of X are occupied by ligands R selected from H, —OH, H₂O, —COO—, CO₃—, NO₃—, SO₄—, PO₄—, —SH, —NH₃, —NH₂, —NH, Cl, Br, and I;
and wherein two or more mercaptophenol units may bond via S—X—S linkages or S—S disulfide linkages to form a dimer, oligomer, or polymer.

9. A complex or derivative of mercaptophenol that is soluble and stable in an aqueous or physiologically relevant medium at a concentration of 1.5-15 mM at 18-25° C. for 12 hours and exhibits antiproliferative, anti-migratory, anti-invasive, and immune-modulatory activity at 0.3-30 μM against malignant or disease-causing cells.

10. The complex of claim 8, having an aqueous solubility at a concentration of 1.5-15 mM at 18-25° C., when dissolved in a medium of pH 5.5-10.0 and remaining stable in such aqueous medium for at least 12 hours.

11. The complex of claim 8, capable of forming a non-covalent conjugate or complex with an amphipathic molecule selected from a nucleotide, an oligonucleotide, a chemotherapeutic nucleoside, a chemotherapeutic nucleotide, a chemotherapeutic oligonucleotide, and polyethylene glycol.

12. The complex or derivative of claim 8, wherein the mercaptophenol molecule comprises two ionizable groups in conjugation, including a thiol group and a phenolic hydroxyl group, and is selected from 2-mercaptophenol, 3-mercaptophenol, and 4-mercaptophenol.

13. The complex or derivative of claim 8, exhibiting antiproliferative, anti-migratory, and anti-invasive activity at a concentration of about 0.3-30 μM against malignant or disease-causing cells.

14. The complex or derivative of claim 8, exhibiting immune-modulatory activity, comprising enhancement of blood-cell production and/or modulation of expression of immune-cell surface markers or receptors at a concentration of about 0.3-30 μM.

15. A process for preparing the compound of claim 1, comprising:
(a) dissolving curcumin in a solvent having a polarity of about 5.0-6.0 selected from methanol, ethanol, propanol, acetonitrile, and acetone;
(b) adding water to the solution of step (a) to obtain a solution having a final water content of about 1-25% (v/v);
(c) adding a metal or metalloid salt selected from a chloride, bromide, iodide, acetate, carbonate, sulfate, nitrate, or phosphate to the solution of step (b) at about 4-50° C. and incubating until a color change and/or turbidity is observed; or
(d) adding an alkaline solution selected from NH₄OH, NaOH, KOH, and Ca(OH)₂ dropwise to the solution of step (b) under stirring at about 4-50° C. until a pH of about 7.5-10.0 is reached, followed by adding the metal or metalloid salt of step (c), and incubating until a color change and/or turbidity is observed; or
(e) adding the metal or metalloid salt of step (c) to the solution of step (b) at about 4-50° C., followed by adding the alkaline solution of step (d) dropwise under stirring until a color change and/or turbidity is observed;
(f) centrifuging the solution or suspension of steps (c)-(e) to obtain a pellet and a supernatant;
(g) drying the pellet and supernatant of step (f) separately under vacuum;
(h) washing the dried pellet and supernatant of step (g) sequentially with solvents of polarity about 0.0-2.5 (n-hexane, toluene, carbon tetrachloride), 3.0-4.4 (dichloromethane, chloroform), 4.5-6.0 (methanol, ethanol, acetonitrile), and 7.5-9.0 (water), and collecting each fraction separately, including pellets;
(i) drying the fractions of step (h) under vacuum; and
(j) isolating the organometallic compound from the dried fractions of step (i) with suitable solvents;
wherein the metal or metalloid salt is added in any of steps (c)-(e) at a molar ratio of curcumin:metal of about 1:0.5 to 1:50.

16. A process for preparing the complex and derivative of claim 8, comprising:
(a) dissolving a mercaptophenol in a solvent having a polarity of about 5.0-6.0 selected from methanol, ethanol, propanol, acetonitrile, and acetone;
(b) adding water to obtain a solution or suspension having a final water content of about 1-25% (v/v);
(c) adding a metal or metalloid salt selected from a chloride, bromide, iodide, acetate, carbonate, sulfate, nitrate, or phosphate at about 4-60° C. and incubating until a color change and/or turbidity is observed; or
(d) adding an alkaline solution selected from NH₄OH, NaOH, KOH, and Ca(OH)₂ dropwise at about 4-60° C., followed by adding the metal or metalloid salt of step (c), and incubating until a color change and/or turbidity is observed; or
(e) adding the metal or metalloid salt of step (c) to the solution of step (b) at about 4-60° C., followed by adding the alkaline solution of step (d) dropwise under stirring until a color change and/or turbidity is observed;
(f) centrifuging the reaction mixtures of step (c)-(e) to obtain a pellet and a supernatant;
(g) drying the pellet and supernatant of step (f) separately under vacuum;
(h) washing the dried pellet and supernatant of step (g) sequentially with solvents of polarity about 0.0-2.5 (n-hexane, toluene, carbon tetrachloride), 3.0-4.4 (dichloromethane, chloroform), 4.5-6.0 (methanol, ethanol, acetonitrile), and 7.5-9.0 (water), and collecting each fraction separately;
(i) drying the fractions of step (h) under vacuum; and
(j) isolating the complex and derivatives from the dried fractions of step (i) with suitable solvents;
wherein the metal or metalloid salt is added in any of steps (c)-(e) at a molar ratio of mercaptophenol:metal of about 1:0.5 to 1:50.

17. A method of treating cancer or another disease in a subject in need thereof, comprising administering to the subject, via parenteral or systemic delivery, a daily dose of about 0.2-2.0 mg/kg of the compound of claim 1, alone or in combination with one or more therapeutic agents, thereby inhibiting proliferation, invasion/migration of disease-causing cells and inducing an immune response.

18. A method of treating cancer or another disease in a subject in need thereof, comprising administering to the subject, via parenteral or systemic delivery or oral ingestion, a daily dose of about 0.5-5.0 mg/kg of the complex or derivatives of claim 8, alone or in combination with one or more therapeutic agents, thereby inhibiting proliferation, invasion/migration of disease-causing cells and inducing an immune response.
